(19) United States
(12) Patent Application Publication
(10) Pub. No.: US 2008/0044348 A1
(43) Pub. Date: Feb. 21, 2008

(54) Tetanus toxin fragment C based imaging agents and methods, and confocal microscopy dataset processes

(76) Inventors: Juri G. Gelovani, Pearland, TX (US); Lucia Gertruida LeRoux, Houston, TX (US); David Schellingerhout, Houston, TX (US); David S. Maxwell, Pearland, TX (US); Main M. Alauddin, Houston, TX (US)

Correspondence Address: BAKER BOTTS, LLP, 910 LOUISIANA, HOUSTON, TX, 77002-4995, US

(21) Appl. No.: 11/771,868

(22) Filed: Jun. 29, 2007

Related U.S. Application Data
(60) Provisional application No. 60/806,375, filed on Jun. 30, 2006.

(51) Int. Cl. A61K 49/00; A61K 49/04; A61K 49/14; C12N 1/20; G06F 17/00

(52) U.S. Cl. 424/1.11; 424/9.1; 424/9.3; 424/9.4; 424/9.6; 435/252.7; 707/104.1; 707/E17.044

(57) ABSTRACT

Methods for purifying Tetanus Toxin Fragment C comprising obtaining a supernatant comprising soluble Tetanus Toxin Fragment C and purifying Tetanus Toxin Fragment C under native conditions to obtain a substantially purified Tetanus Toxin Fragment C. Imaging agents comprising a Tetanus Toxin Fragment C and a reporter, and methods thereof. Methods comprising processing confocal microscopy datasets to provide a 360 degree average fluorescence intensity profile from the center of a spheroid towards the outer edge of the spheroid.

### CROSS-REFERENCE TO RELATED APPLICATIONS

This application claims the benefit of U.S. Provisional Application Ser. No. 60/806,375 filed on Jun. 30, 2006, which is incorporated by reference.

### BACKGROUND

Purification of proteins from a heterogeneous mixture often involves a multi-step process that makes use of the physical, chemical, and electrical properties of the protein being purified. Important properties of a protein that are relevant to its purification are (a) solubility, which determines the ability of the protein to remain in solution or to precipitate out in the presence of salt; (b) charge, which is an important property relevant to ion exchange chromatography and isoelectric focusing; (c) size, which is relevant in processes involving dialysis, gel-filtration chromatography, gel electrophoresis and sedimentation velocity; (d) specific binding, which allows purification of a protein based on its binding to a ligand; and (e) ability to form complexes in the presence of other reagents, such as in antibody precipitation. Protein detection and purification has become a major focus of research activities in view of the challenges faced by researchers involved in functional genomics and proteomics.

Tetanus toxin fragment C (TTC) is a 50 kD non-toxic polypeptide that is one of the products of cleavage of tetanus toxin by papain. Previous studies indicates that TTC in all its forms is highly insoluble and difficult to purify without resorting to denaturing condition. Denaturing conditions include the use of 6M Guanidine Chloride or 6-8 M Urea for solubilization of protein inclusion bodies post bacterial pellet suspension in 20 mM Tris-HCL (pH 8) and lysation with a French Press. Protein purification under denaturing conditions unfolds TTC and linearizes the 3-dimensional structure needed for biological activity. Protein refolding from this linearized form is difficult, but can be accomplished by means of a multistep dialysis with a gradual decrease in amount of denaturing agent. The refolding process is complex and not always successful.

Nerve function may be evaluated using electrophysiology/electromyography (EMG) EMG is painful and invasive; most patients do not tolerate it well. EMG is limited in what nerves it can evaluate, and can for example, not evaluate the spinal cord's function itself directly because of the need for stimulating and sensing needles to be inserted proximally and distally into the neuromuscular or neurosensor units being investigated.

### SUMMARY

The present disclosure, according to specific example embodiments, generally relates to protein purification and imaging. In particular, the present disclosure relates to a Tetanus Toxin Fragment C (TTC) based imaging agent and associated methods of use, as well as methods to process confocal microscopy datasets. The TTC based imaging agents of the present disclosure generally comprise a Tetanus Toxin Fragment C and a reporter, and such imaging agents may be useful diagnostically, for example, as a means of investigating nerve diseases of various types.

The present disclosure, according to specific example embodiments, also provides methods comprising processing confocal microscopy datasets to provide a 360 degree average fluorescence intensity profile from the center of a spheroid towards the outer edge of the spheroid. Such methods, among other things, allows for quantitative characterization of spatial heterogeneity and temporal dynamics of fluorescence distribution within multi-cellular 3D spheroids.

### DRAWINGS

Some specific example embodiments of the disclosure may be understood by referring, in part, to the following description and the accompanying drawings.

FIG. 1 shows Western Immuno-detection with anti-TTC. Lane 1 shows (1 ul) 2 ug Roche TTC, lane 2 shows native conditions-10 ul supernatant 1 after bacterial lysis, lane 3 shows denaturing conditions-10 ul pellet 2 (redissolved in 10 ml buffer), and lane 4 shows denaturing conditions-10 ul supernatant 2.

FIG. 2 shows an SDS page gel of TTC solubilized bacterial fraction in denaturing conditions with lane 1 initial fraction, lane 2 unbound after Ni bead addition, lane 3 5 ul TTC elution, lane 4 10 ul TTC elution, lane 5 1 ul (2 ug) Roche TTC, and lane 6 20 ul Ni beads post washing.

FIG. 3 shows purification of the TTC solubilized bacterial fraction in denaturing conditions, post dialysis to a Tris Buffer pH 8. Lane 1 2 ug Roche TTC (1 ul) (*), lane 2 1 ul Pre-dialyzed TTC, lane 3 1 ul Dialyzed A37 TTC(0.3M Tris Buffer pH 8), lane 4 2 ul Dialyzed A37 TTC(0.3M Tris Buffer pH 8), lane 5 3 ul Dialyzed A37 TTC(0.3M Tris Buffer pH 8), lane 6 4 ul Dialyzed A37 TTC(0.3M Tris Buffer pH 8), lane 7 5 ul Dialyzed A37 TTC(0.3M Tris Buffer pH 8), and lane 8 10 ul Dialyzed A37 TTC(0.3M Tris Buffer pH 8). Approximated concentration of A37 is 0.6 ug/ul.

FIG. 4 shows purification of TTC using the natively solubilized bacterial fraction. Lane 1 shows 5 ul Marker, lane 2 shows 10 ul A37 pellet dissolved in PBS, lane 3 shows 10 ul Initial A37, land 4 shows 10 ul Unbound A37 (purification on A40), lane 5 shows 20 ul beads, lane 6 shows A37 frozen sample on Dec. 28, 2005, run on Jan. 09, 2006, lane 7 shows A37 pre-dialyzed, purified Dec. 28, 2005, and lane 8 shows 1 ul (2 ug) Roche TTC.

FIG. 5 shows an SDS PAGE gel of Alexa680-TTC. Lane 1 shows 5 ul Molecular weight standard, lane 2 shows Tug TTC Roche, lane 3 shows 2 ug TTC Roche, lane 4 shows 3 ug TTC Roche, lane 5 shows 1 ul Tris-Chelate TTC (2.4 ug/ul), and lane 6 shows 2 ul AlexaFluorTTC fraction 1 (1.2 ug.ul).

FIG. 6 shows Western Anti-TTC immuno detection. Lane 1 shows 2 ug TTC before labeling, lane 2 shows 2 ug Alexa Fluor labeled TTC, lane 3 shows 2 ug TTC Roche (positive control), and lane 4 shows 2 ug BSA (negative control).

FIG. 7 shows an IVUS 200 scan of the SDS-PAGE gel of Alexa680-TTC (CY5.5 filter set) and associated Coomasie blue stain of the gel.

FIG. 8 shows PC12 cells after 4 h incubation with Alexa-TTC.

FIG. 9 shows TTC in the right sciatic nerve 5 hours after TTC injection into a mouse under a Xenogen fluorescent imager with a GFP filter.

FIG. 10 shows HSA in the left sciatic nerve 5 hours after TTC injection into a mouse under a Xenogen fluorescent imager with a CY5.5 filter.

FIG. 11 shows HSA in the left sciatic nerve 5 hours after TTC injection into a mouse under a Xenogen fluorescent imager with a DSRed filter.

FIG. 12 shows HSA (red) in the left calf and TTC (green) in the right calf of a mouse and along the sciatic nerve of a mouse imaged with a Xenogen fluorescent imager 45 minutes after injection into the gastrocnemius muscle.

FIG. 13 shows TTC (green) in the right sciatic nerve trifurcation of a mouse imaged with a Xenogen fluorescent imager 80 minutes after injection into the gastrocnemius muscle.

FIG. 14 shows TTC (green) in the right sciatic nerve trifurcation of a mouse imaged with a Xenogen fluorescent imager 90 minutes after injection into the gastrocnemius muscle.

FIG. 15 shows TTC (green) in the right sciatic nerve trifurcation of a mouse imaged with a Xenogen fluorescent imager 110 minutes after injection into the gastrocnemius muscle.

FIG. 16 shows TTC (green) in the right sciatic nerve trifurcation of a mouse imaged with a Xenogen fluorescent imager 4 hours and 20 minutes after injection into the gastrocnemius muscle.

FIG. 17 shows TTC (green) in the excised right sciatic nerve of a mouse imaged with a Xenogen fluorescent imager 5 hours after injection into the gastrocnemius muscle, with background fluorescence only in the left sciatic nerve.

FIG. 18 shows diffuse TTC (green) in the right calf of a mouse imaged with a Xenogen fluorescent imager 23 hours after injection into the gastrocnemius muscle and no HSA fluorescence in the left calf.

FIG. 19 shows granular TTC (green) distribution in the right calf of a mouse imaged with a Xenogen fluorescent imager 24 hours after injection into the gastrocnemius muscle.

FIG. 20 shows TTC (green) in excised sciatic nerves of a mouse imaged with a Xenogen fluorescent imager 24 hours after injection into the gastrocnemius muscle.

FIG. 21 shows TTC (green) distribution in the right calf of a mouse imaged with a Xenogen fluorescent imager 60 minutes after injection into the gastrocnemius muscle.

FIG. 22 shows TTC (green) distribution in the right calf of a mouse imaged with a Xenogen fluorescent imager 36 hours after injection into the gastrocnemius muscle.

FIG. 23 shows a second view of TTC (green) distribution in the right calf of a mouse imaged with a Xenogen fluorescent imager 36 hours after injection into the gastrocnemius muscle.

FIG. 24 shows an image of the animal subject 24 hours after injection of Alexa680-TTC into the right hind leg, with skin off.

FIG. 25 shows an image of the animal subject 24 hours after injection of Alexa680-TTC into the right hind leg, with nerve open.

FIG. 26 shows an image of the animal subject 24 hours after injection of Alexa680-TTC into the right hind leg, with spine open.

FIG. 27 shows an image of the animal subject 24 hours after injection of Alexa680-TTC into the right hind leg, with nerves dissected.

FIG. 28 shows an image of the blank chamber.

FIG. 29 shows images from an in vivo time course study over a period of 12 hours in C57BL/6 mice.

FIG. 30 shows an image of the C57Bl/6 mouse 6 hours after treatment with Alexa 680-TTC.

FIG. 31 shows images of excised muscles on different backgrounds.

FIG. 32 shows an SDS-PAGE of the TTC-His protein after EC conjugation, with appropriate standards. Lane 1: 1 ug TC-Roche standard. Lane 2: 2 ug TC-Roche standard. Lane 3: 3 ug TC-Roche standard. Lane 4: 1 ul TC-His (A122). Lane 5: 2 ul TC-His (A122). Lane 6: 1 ul TC-His-EC (A122). Lane 7: 2 ul TC-His-EC (A122). Lane 8: 3 ul TC-His-EC (A122).

FIG. 33 shows immunodetection of TTC-His-EC with appropriate standards. Lane 1: 2 ug TC Roche. Lane 2: 2 ug TC-HIS A122. Lane 3: 2 ug TC-His-EC A122. Lane 4: 2 ug HSA.

FIG. 34 shows the results of an ELISA of TC-His-EC conjugates, as well as TC-Roche positive control, TC-His conjugate reference and HSA standard.

FIG. 35 shows PC12 cell uptake of Alexa488-TC-His without fixation of the cells.

FIG. 36 shows PC12 cell uptake of Alexa488-TC-His after fixation of the cells.

FIG. 37 shows PC12 cell uptake of Alexa488-TC-His after fixation of the cells.

FIG. 38 shows PC12 cell uptake of Alexa488-TC-His after fixation, washing, and antibody staining of the cells.

FIG. 39 shows PC12 cell uptake of Alexa488-TC-His after fixation, washing, and antibody staining of the cells.

FIG. 40 shows an ultraviolet quantitation of PC12 uptake.

FIG. 41 shows immunoreactivity of A37 conjugate from ELISA response.

FIG. 42 shows ELISA assay results for conjugate with and without indium.

FIG. 43 shows Coomasie blue staining of the gel of TTC protein labeled with DOTA chelator.

FIG. 44 shows Western blot of TTC protein labeled with DOTA chelator.

FIG. 45 shows Ponceau Red staining of the gel of TTC labeled with DOTA chelator.

FIG. 46 shows thin layer liquid chromatography analysis (TLC) using 80:20 MetOH Water on Cellulose of TTC-DOTA-Indium-111.

FIG. 47 shows analysis of TTC-DOTA-Indium-111 using saline TLC on cellulose.

FIG. 48 shows the pH dependence of DOTA-Indium chelation by assessment of cellulose-saline TLC.

FIG. 49 shows optimization of Indium-Acetate (citrate) weakly chelated species in solution as a function of pH and time.

FIG. 50 shows cellulose-saline TLC after 30 minute incubation of Indium-Acetate at stated pH with Tris with or without DOTA.

FIG. 51 shows dose calibrator measurement and gamma counter measurement of binding of TTC-DOTA to In-Acetate (pH 5 preparation).

FIG. 52 shows MCAM imaging procedure.

FIG. 53 shows a coded aperture of the imaging procedure.

FIG. 54 shows the dissection procedure involving dissection of the sciatic nerve.

FIG. 55 shows the dissection procedure involving dissection of the sciatic nerve.

FIG. 56 shows the dissection procedure for exposing the spinal cord.

FIG. 57 shows a histogram of dissected nerve weights.

FIG. 58 shows an image of a mouse subject at time point 0 hours after injection.

FIG. 59 shows an image of a mouse subject 8 hours after injection, indicating activity along the nerve.

FIG. 60 shows an image of a mouse subject 24 hours after injection.

FIG. 61 shows an image of a mouse subject 27 hours after injection, indicating activity along the nerve.

FIG. 62 shows an image of a mouse subject 28 hours after injection, indicating activity along the nerve.

FIG. 63 shows an image of a mouse subject from a different view 28 hours after injection.

FIG. 64 shows an image of a mouse subject 30 hours after injection, indicating activity along the nerve.

FIG. 65 shows an image of a mouse subject 48 hours after injection.

FIG. 66 shows biodistribution of TC-DOTA-In111 after gastrocnemicus injection, assessed per organ as a function of percentage of ID/gram after 4, 24, and 72 hours.

FIG. 67 shows biodistribution of TC-DOTA-In111 after gastrocnemicus injection, assessed per organ as a function of percentage of ID/gram after 4, 24, and 72 hours.

FIG. 68 shows right-left rations of TTC-DOTA-In111 activity at 4, 24, and 72 hours in the nerves and in the legs.

FIG. 69 shows an excretion profile of TTC-DOTA-In111.

FIG. 70 shows a CellVizio image of the gastrocnemius muscle of a C57BL6 mouse under isofluorane anesthesia at several timepoints after a 15 uL dose of Alexa488-TTC into the gastrocnemius muscle.

FIG. 71 shows a CellVizio image of the sciatic nerve bitruncation of a C57BL6 mouse under isofluorane anesthesia at several timepoints after a 15 uL dose of Alexa488-TTC into the gastrocnemius muscle.

FIG. 72 shows a CellVizio image of the gastrocnemius muscle of a C57BL6 mouse under isofluorane anesthesia at several timepoints after a 50 uL dose of Alexa488-TTC into the gastrocnemius muscle.

FIG. 73 shows a CellVizio image of the sciatic nerve bitruncation of a C57BL6 mouse under isofluorane anesthesia at several timepoints after a 50 uL dose of Alexa488-TTC into the gastrocnemius muscle.

FIG. 74 shows a CellVizio image at and near the Junction of the sciatic nerve and the gastrocnemius muscle of a C57BL6 mouse under isofluorane anesthesia at several timepoints after a 15 uL dose of Alexa488-TTC into the gastrocnemius muscle.

FIG. 75 shows a Xenogen fluorescent imager image of the gastrocnemius muscle of a C57BL6 mouse under isofluorane anesthesia at several timepoints after a 15 uL dose of Alexa488-TTC into the gastrocnemius muscle.

FIG. 76 shows a CellVizio image at and near the junction of the sciatic nerve and the gastrocnemius muscle of a C57BL6 mouse under isofluorane anesthesia at 72 and 96 hours after a 50 uL dose of Alexa488-TTC into the gastrocnemius muscle.

FIG. 77 shows an image from a Xenogen fluorescent imager of excised sciatic nerves from C57BL6 mice collected at various timepoints.

FIG. 78 shows an image from a Xenogen fluorescent imager of excised sciatic nerves from C57BL6 mice collected at various timepoints.

FIG. 79 shows Western blotting and immunodetection of chelated and unchelated TTC stored under a variety of conditions. Lane 1: 2 ug TTC (A79) at 4 degrees Celsius. Lane 2: 2 ug TTC (A79) at 25 degrees Celsius. Lane 3: 2 ug TTC (A79) stored for 23 hours at 4 degrees Celsius then for 1 hour at 37 degrees Celsius. Lane 4: 2 ug TTC (A79) stored for 23 hours at 4 degree then for 1 hour at 43 degrees Celsius. Lane 5: 2 ug TTC (A79) stored for 23 hours at 25 degrees Celsius then for 1 hour at 37 degrees Celsius. Lane 6: 2 ug TTC (A79) stored for 23 hours at 25 degrees Celsius then for 1 hour at 43 degrees Celsius. Lane 7: 2 ug 200:1 TTC after DOTA chelation stored at 4 degrees Celsius. Lane 8: 2 ug 100:1 TTC after DOTA chelation stored at 4 degrees Celsius. Lane 9: 2 ug 200:1 TTC after DOTA chelation stored at 25 degrees Celsius. Lane 10: 2 ug 100:1 TTC after DOTA chelation stored at 25 degrees Celsius Lane 11: 2 ug Roche TTC positive control. Lane 12: 2 ug BSA standard.

FIG. 80 shows the results of an ELISA of chelated and unchelated TTC samples stored under a variety of temperature conditions over a 24-hour period.

FIG. 81 shows Western blotting and immunodetection of chelated and unchelated TTC stored under a variety of conditions. Lane 1: 2 ug TTC (A78) at 4 degrees Celsius. Lane 2: 2 ug TTC (A78) at 25 degrees Celsius. Lane 3: 2 ug TTC (A78) stored for 23 hours at 4 degrees Celsius then for 1 hour at 37 degrees Celsius. Lane 4: 2 ug TTC (A78) stored for 23 hours at 4 degree then for 1 hour at 43 degrees Celsius. Lane 5: 2 ug TTC (A78) stored for 23 hours at 25 degrees Celsius then for 1 hour at 37 degrees Celsius, Lane 6: 2 ug TTC (A78) stored for 23 hours at 25 degrees Celsius then for 1 hour at 43 degrees Celsius. Lane 7: 2 ug Roche TTC positive control. Lane 8: 2 ug BSA standard.

FIG. 82 shows the results of an ELISA of TTC samples stored under a variety of temperature conditions over a 24-hour period.

FIG. 83 shows an image of uptake of TTC by PC12 cells mounted with Molecular Probes anti-fading medium from a Confocal FV 1000 microscope with FitC laser power 565.

FIG. 84 shows an image of uptake of TTC by PC12 cells mounted with Molecular Probes anti-fading medium from a Confocal FV 1000 microscope with FitC laser power 465.

FIG. 85 shows sample confocal microscopy images showing central 2D sections of the same spheroid with different color fluorescence.

FIG. 86 shows a flowchart of spheroid analysis algorithm.

FIG. 87 shows a screen capture from ImageJ Session Running v1.4 of Spheroid Analysis Macro on images shown in FIG. 87. Several dialogs were removed and the RFP image was reloaded after macro completed.

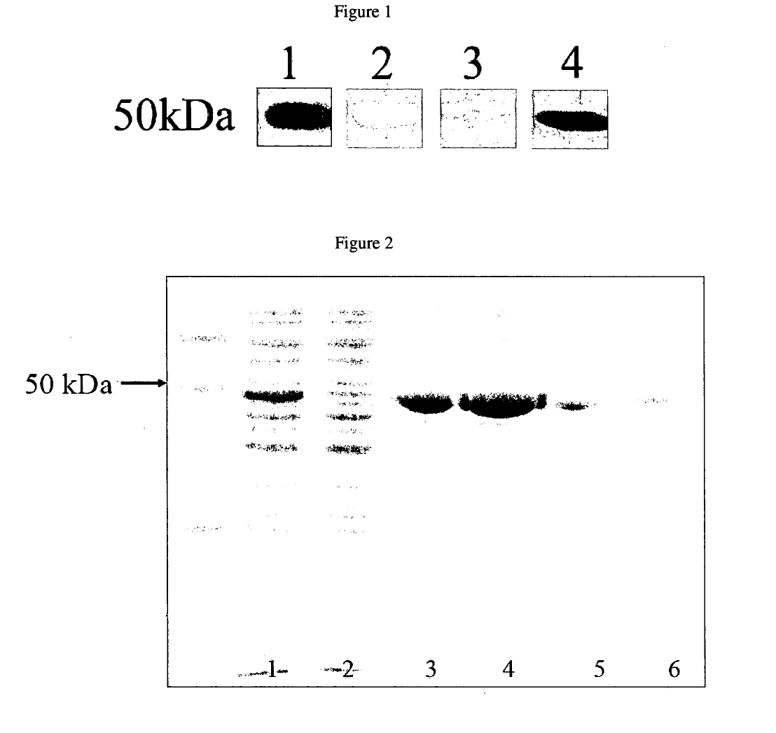

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the drawings and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to certain embodiments, provides methods for purifying TTC comprising obtaining a supernatant comprising soluble TTC and purifying TTC from the supernatant under native conditions to obtain a substantially purified TTC. Such methods may avoid denaturation of TTC, and thus may preserve the biologically active conformation of TTC. In certain embodiments, the TTC may be His-tagged, and such His-tagged TTC may be purified using a column based purification kit, for example, nickel coated sephadex beads and imidazole.

The present disclosure, according to certain embodiments, provides imaging agents comprising TTC and a reporter. Such imaging agents may allow imaging the process of retrograde axonal transport, among other things. The TTC in the imaging agent may be the complete TTC protein or fragment thereof, so long as it retains biological activity. In this context, biological activity may refer to the properties of neuronal uptake and retrograde transport, which TTC possesses. The TTC is associated with a reporter to allow the detection of TTC activity (e.g., neuronal uptake and retrograde transport). The reporter may be any molecule that produces signal detectable by various non-invasive and invasive imaging technologies. Examples of reporters include fluorescent labels and radiolabels such as, for example, Alexa fluors, fluorescent dyes, green fluorescent proteins, red fluorescent proteins, Alexa dyes, and indium. Imaging technologies that may be used in conjunction with the imaging agents of the present disclosure, include, but are not limited to, magnetic resonance imaging (MRI), positron emission tomography (PET), and computed tomography (CT). In certain embodiments, the imaging agent of the present disclosure may be adapted to carry not only a reporter, but instead or in addition, a therapeutic moiety such as a drug, growth factor, radiation emitting compound or the like, allowing the compound to be used for therapeutic purposes in addition to, or instead of diagnostic applications. Accordingly, imaging agents of the present disclosure may be used in in methods for imaging retrograde axonl transport and methods to detect and/or treat a variety of peripheral nerve diseases. In these methods, the imaging agent may be injected into a mammal and a signal may be detected.

The present disclosure also provides, according to certain embodiments, a methods for processing confocal microscopy datasets to provide a 360 degree average fluorescence intensity profile from the center of spheroid towards the outer edge of the spheroid. As used herein, the term “spheroids” refers to three-dimensional aggregates of cells that serve as in vitro models of tumors, and model cancerous processes more closely than do monolayer cultures of cancer cells. In certain embodiment, spheroid refers to other cells, tissues, or cell-tissue constructs of biological relevance could be studied with similar strategies incorporating fluorescent reporters and suitable promoters in conjunction with the methods of the present disclosure. In certain embodiments, the cells of interest may be a portion of a tumor spheroid. In certain other embodiments, any compound comprising a reporter may be studied using the methods to process confocol microscopy datasets.

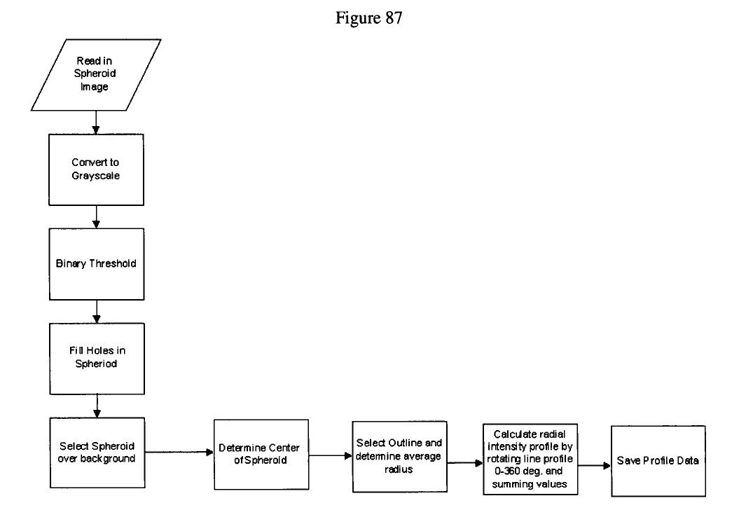

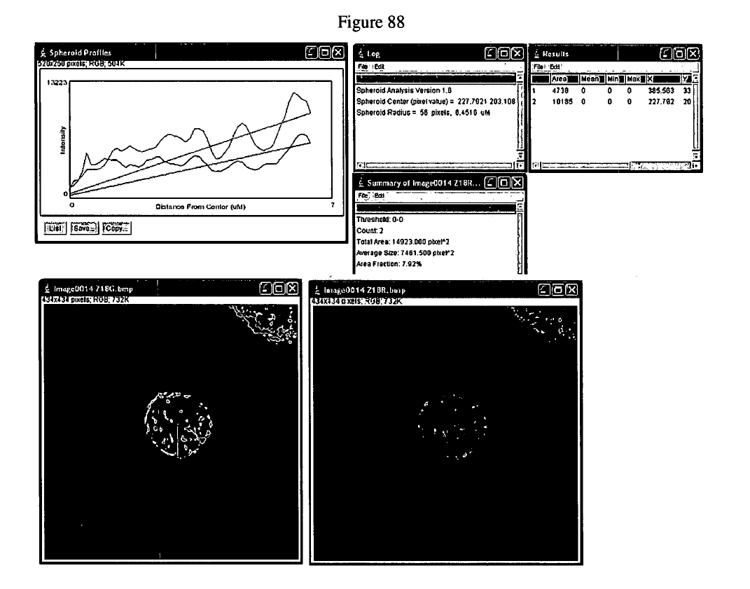

In one embodiment, an average radial profile image analysis on a user specified central image slice through the spheroid may be performed. The RFP channel may be used to threshold the data and to determine the center of the spheroid. Using this computer determined center as a fulcrum, a radial arc was swept through user specified 360 degrees, while plotting an expression plot profile along each radius (plot line thickness=1 pixel) from a reporter (e.g., a fluorescent reporter). Such methods may be used to analyze the large image datasets of spheroids and automatically determine the center, radius, and radial intensity profile of a spheroid. Profiles generated as a result of various experimental conditions may be analyzed with this method in this manner with minimal user interaction. The flow chart (FIG. 87) describes one example of an process that may be used in conjunction with the methods of the present disclosure, which may be implemented using a computer that includes at least one processor and a memory.

In certain embodiments, the methods of the present disclosure may be a macro in software. In certain other embodiments, the methods of the present disclosure may be implemented as a separate image analysis program, or as a component of a larger image analysis software platform.

One example of a method of the processing confocal microscopy datasets may be executed in the form of a macro. For example, the text of a working macro that works with v1.35s of the ImageJ program as obtained from http://rsb.info.nih.gov/ij/ if provided below. This macro serves to demonstrate a working implementation of one example for processing confocal microscopy datasets:

// The purpose of this ImageJ script is to automate the process of analyzing
the
// spheroids. The macro finds the center of the spheroid, the average
radius,
// then sums up the profile around the spheroid.
// V1.6 by David S. Maxwell
// UTMDACC
programversion = 1.6;
print(“Spheroid Analysis Version”,programversion);
//
// Version History
// v1.6 2007-05-22 - Handle additional background particles and only selects
nearest to center of image as spheroid to analyze. Added checkbox to close
images at end of analysis.
// v1.5 2007-05-22 - Closed any open images at end of script
// v1.4 2006-05-18 - Corrected problems with threshold by allowing it to be
manually set
// v1 [symbol] 3 2006-04-17 - Added ability for user to change low end of circularity
// v1 [symbol] 2 Change low end of circularity to 0.4 (from 0 [symbol] 5)
// v1 [symbol] 1 Fixed bug with doWand
// V1.0 Added save at end of macro, converted distances to uM, allowed
variable theta
// V0.9 First version distributed for testing
//
// Rough Outline of steps taken in macro
//
// Install and run macro
// Open dialog to set directory
// Select red spheroid and green spheroid files
// Open dialog to modify defaults (size conversion, minimum circularity,
// angle change for rotating profile)
// Read in red sheroid
// Binary Threshold
// Binary Dilate for 7 steps to fill holes
// Binary Erode for 7 steps to return back to normal size
// Analyze for particle size >=500 and circularity >=0.35
// Select one particle that is closest to center of image from the list of
// possible particles
// Determine center of spheroid and graphically form outline of spheroid
// Select outline of spheroid
// Determine avg. radius from measuring distance from points on outline
// to center of spheroid
// Form line from center to avg. radius, rotate by theta and get line
// profile, summing the profile in the process
// Open green spheroid
// Process profile in same manner as red spheroid, except use the center
// and avg. radius from red spheroid
// Save out profiles for both spheroids
// Import data to graphing program.
// Defaults for program
// changetheta determines the stepping size around the circle
// (i.e. resolution)
// imageSize is the size (in uM) equivalent to image height in pixels
// mincircularity sets the minimum value below which will not be
// considered during the analyze particle stage
// minthreshold and maxthreshold determine the values used for thresholding
changetheta = 1.0;
imageSize = 50;
mincircularity = 0.350;
minthreshold = 11;
maxthreshold = 85;
// Returns the maximum value found in an array
function maxArray(a) {

maxvalue = −100000;
for (i=0; i<a [symbol] length; i++) {

if (a[i] > maxvalue) {

maxvalue = a[i];

}

}
return maxvalue;

}
// Returns the distance between two points in x,y space
function xydist(x1, y1, x2, y2) {

diffx = x2 − x1;
diffy = y2 − y1;
distance = sqrt(diffx*diffx + diffy*diffy);
return distance;

}
// Open up a dialog to select the directory (not the file)
dir = getDirectory(“Choose a Directory ”);
filesInDir = getFileList(dir);
// Function to handle opening a file from a list of files
function getDirFiles(choiceText) {

Dialog.create(“Open Files”);
Dialog.addChoice(choiceText,filesInDir);
Dialog.show( );
choice=Dialog.getChoice( );
return choice;

}
chosenFile = getDirFiles(“Red Spheroid:”);
chosenFile2 = getDirFiles(“Green Spheroid:”);
//chosenDir=getDirectory(“”);
open(dir+chosenFile);
// Determine center of image in term of pixels
imageHeight = getHeight( );
imageWidth = getWidth( );
imageCenterX = round(imageHeight / 2.0);
imageCenterY = round(imageWidth / 2.0);
Dialog.create(“Defaults”);
Dialog [symbol] addNumber(“Image size in uM:”, 50);
Dialog [symbol] addNumber(“Theta Resolution:”, changetheta);
Dialog [symbol] addNumber(“Minimum Circularity:”, mincircularity);
Dialog.addNumber(“Minimum Threshold:”, minthreshold);
Dialog.addNumber(“Maximum Threshold:”, maxthreshold);
Dialog.addCheckbox(“Close Images after analysis”, true);
Dialog.show( );
imageSize = Dialog.getNumber( );
changetheta = Dialog.getNumber( );
mincircularity = Dialog.getNumber( );
minthreshold = Dialog [symbol] getNumber( );
maxthreshold = Dialog [symbol] getNumber( );
CloseImages = Dialog.getCheckbox( );
// Setup measurement correctly, so center is written when Analyze is done
pi = 3.14159265
angletorad = 2*pi/360.
setLineWidth(5);
run(“Set Measurements...”, “area mean min centroid area_fraction
redirect=None decimal=3”);
// The following works to handle the thresholding in difficult cases
// Previous to this, the setAutoThreshold was used, but it failed in some
cases
run(“8-bit”);
//setThreshold(8,65,“black & white”);
setThreshold(minthreshold,maxthreshold,“black & white”);
run(“Threshold”, “thresholded remaining black”);
// The following sort of fills in holes in the spheroid and then goes back to
normal size
// This makes the measurement part easier
for (i=1; i<=7; i++) {

run(“Dilate”);

}
for (i=1; i<=7; i++) {

run(“Erode”);

}
// Analyze the particle(s)
// Generally, only one particle is seen having the size and circularity, but
// sometimes it finds more than one. When this happens, the one closest to
// the image center is selected and processed.
run(“Analyze Particles...”, “size=500-Infinity
circularity=”+mincircularity+“−1.00 show=Outlines display summarize record”);
currentRow = 0;
bestRow = currentRow;
minDistCenter = 999999.0;
while (currentRow < nResults) {

x = getResult(“X”, currentRow);
y = getResult(“Y”, currentRow);
distCenter = xydist(x, y, imageCenterX, imageCenterY);
if (distCenter < minDistCenter) {

minDistCenter = distCenter;
bestRow = currentRow;

}
currentRow = currentRow + 1;

}
// x and y are the center of the spheroid
x = getResult(“X”,bestRow);
y = getResult(“Y”,bestRow);
moveTo(x,y);
//lineTo(0,0);
// Do a wand selection, which basically selects the displayed outline
doWand(x+10,y+10);
getSelectionCoordinates(a,b);
// Go through and find the avg. radius based on the points defining the
outline
Sumradius = 0.0;
for (i=0; i<a.length; i++) {

radius = xydist(a[i], b[i], x, y);
sumradius = sumradius + radius;

}
avgradius = round(sumradius / a [symbol] length);
print(“Spheroid Center (pixel value) = ”, x, y);
close( );
close( );
open(dir+chosenFile);
// Generate an array slightly larger than the determined avg. radius,
// because the profile seems to vary a bit as it goes around the circle
sizeprofile = avgradius + 5;
sumprofile = newArray(sizeprofile);
sumprofile2 = newArray(sizeprofile);
distFromCenter = newArray(sizeprofile);
//
for (theta=0.0; theta<=360.0; theta=theta+changetheta) {

circy = cos(theta*angletorad) * avgradius;
circx = sin(theta*angletorad) * avgradius;
transx = x + circx;
transy = y + circy;
makeLine(x,y,transx,transy);
// run(“Plot Profile”);
profile = getProfile( );
for (i=0; i<profile [symbol] length; i++) {

sumprofile[i] = sumprofile[i] + profile[i];

}
//wait(2);

}
close( );
open(dir+chosenFile2);
for (theta=0.0; theta<=360.0; theta=theta+changetheta) {

circy = cos(theta*angletorad) * avgradius;
circx = sin(theta*angletorad) * avgradius;
transx = x + circx;
transy = y + circy;
makeLine(x,y,transx,transy);
// run(“Plot Profile”);
profile = getProfile( );
for (i=0; i<profile.length; i++) {

sumprofile2[i] = sumprofile2[i] + profile[i];

}
//wait(2);

}
print(“Spheroid Radius = ”, avgradius, “ pixels, ”,
avgradius*(imageSize/imageHeight), “ uM”);
// Generate an array containing converted distances
for (i=0; i<profile.length; i++) {

distFromCenter[i] = i * (imageSize/imageHeight);

}
max1 = maxArray(sumprofile);
max2 = maxArray(sumprofile2);
ymax = max2;
if (max1 > max2) {

ymax = max1;

}
xmax = maxArray(distFromCenter);
// Set the y axis maximum a little higher than maximum value
graphYmax = round(1.1 * ymax);
graphXmax = round(1.1 * xmax);
Plot.create(“Spheroid Profiles”, “Distance From Center (uM)”, “Intensity”);
Plot.setLimits(0, graphXmax, 0, graphYmax);
Plot.setColor(“red”);
Plot.add(“line”, distFromCenter, sumprofile);
Plot.setColor(“green”);
Plot.add(“line”, distFromCenter, sumprofile2);
Plot.show( );
// Close any left-over open images
if (CloseImages == 1) {

while (nImages >= 1) {

close( );

}

}
// Open up dialog to save data from spheroid profile
fileOut = File.open(“”);
for (i=0; i<profile.length; i++) {

print(fileOut, distFromCenter[i] + “ ” + sumprofile[i] + “ ” +

sumprofile2[i]);
}
File.close(fileOut);

In one specific embodiment, the profiles of a spheroid comprised of cells expressing both Red Fluorescent Protein (RFP) under control of a constitutive CMV promoter and Green Fluorescent Protein (GFP) under control of a dxBE (Hypoxic Responsive Element) promoter are compared and have utility as a model of hypoxia in tumor cells. For example, an algorithm may be used for the analysis of biochemical events (in this case hypoxia as a function of distance from the center of the spheroid) in 3D space in a quantitative semi-automatic manner. The methods of the present disclosure allow analysis of these complex data.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### EXAMPLES

Purification of TTC

Three bacterial pellets were combined and induced with 1 mMIPTG at OD 0.6 at 30° C. The pellets were solubilized with 0.1 mg/mL Lysozyme in 20 mM Tris-HCL+500 mM NaCl. Pellets were stirred for 1 hour at room temperature and this fraction was analyzed for solubilized TTC in native conditions. The fraction was sonicated 30 sec (3 times) with 60 sec breaks and then Spun at 8000 g for 20 minutes (clear post lysis supernatant+pellet). The supernatant and the small pellet were analyzed after denaturing conditions Denaturing conditions refers to exposing the inclusion body pellet to Urea for 3 hours, and spun down at 8000 g for 20 min, purify using standard methods with His-Nickel coated beads. Native conditions refer to natively collected supernatant fraction purified using standard methods with His-Nickel coated beads.

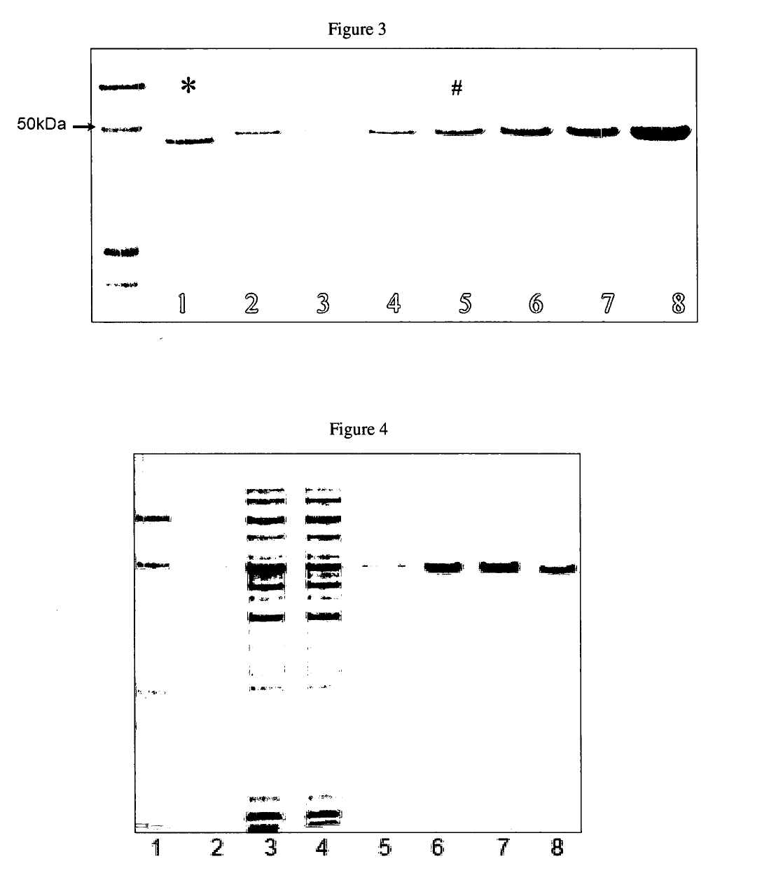

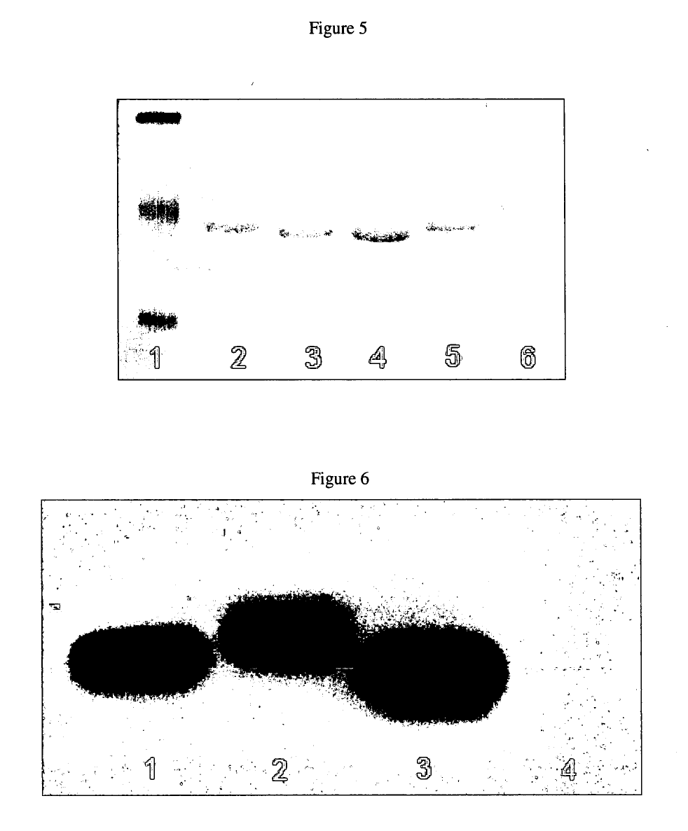

As shown in FIG. 3, the TTC protein is present in the bacterially lysed supernatant in native conditions (lane 2) and both in the pellet (lane 3) and supernatant fraction of post solubilized inclusion bodies in denaturing conditions. FIG. 4 shows purification of the TTC solubilized bacterial fraction in denaturing conditions. FIG. 5 shows purification of the TTC solubilized bacterial fraction in denaturing conditions, post dialysis to a Tris Buffer pH 8. FIG. 6 shows purification of TTC using the natively solubilized Bacterial fraction. This example shows that TTC can be purified using native conditions.

TTC Fluorescent Labeling

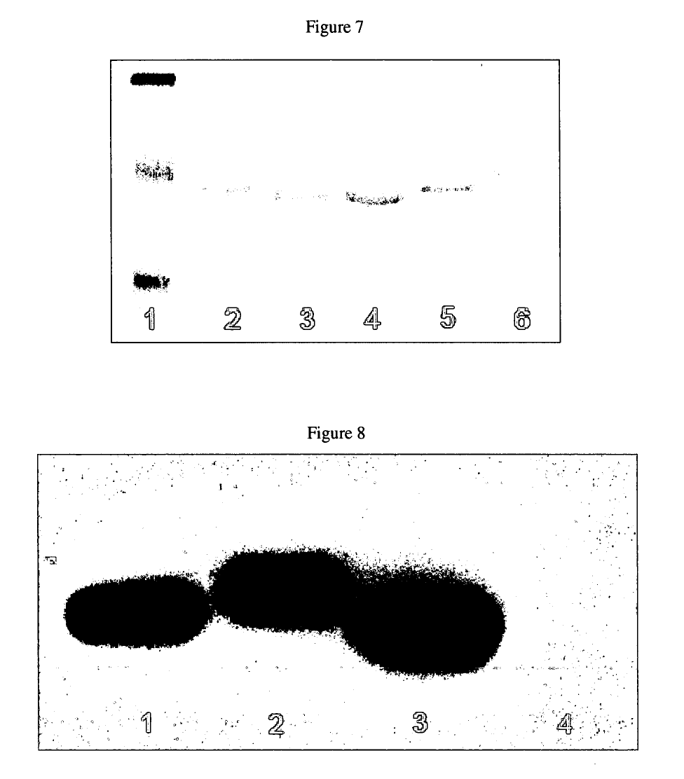

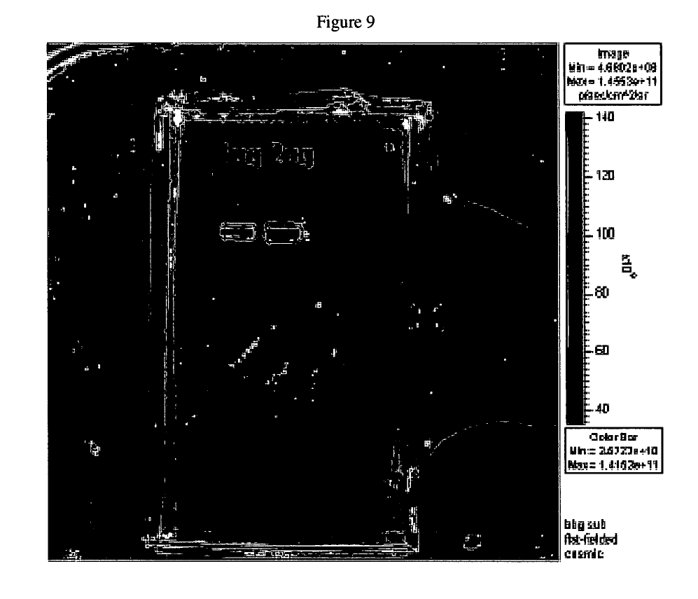

To label TTC and demonstrate retention of biological activity of the compound, an Alexa fluor 680 protein labeling kit was used (Molecular probes-A20172). Purified TTC was labeled with initial concentration of 2 mg/ml (500 ul). 50 ul of 1M NaCO3 buffer to TTC. The total fraction of TTC (550 ul) was placed over column. Collection light blue band, 30 min after application. 3 fractions were collected and analyzed (FIG. 7). Western Anti-TTC immunodetectlon was performed (FIG. 8). An IVUS 200 used to scan the SDS-PAGE gel of Alexa680-TTC (CY5.5 filter set). A clear fluorescent signal was associated with protein (FIG. 9).

Agent to Image Retrograde Axonal Transport

The TTC plasmid DH5 alpha competent cells were subcloned and the sequenced DNA was similar to the published sequence. Protein expression and purification was performed in Epicurian Coli BL31 DE3 using standard methods. The purity and integrity of the protein was analyzed by sodium dodecyl sulfate-polyacrylamide gel electrophoresis (SDS-PAGE). The immunoreactivity of the TTC protein was confirmed via Western blotting and ELISA assays using a mouse monoclonal antibody to the C-fragment of tetanus toxin (Roche #11 131 621 001). The integrity and immunoreactivity of the Tetanus toxin C protein and the derivatives we have prepared remained constant. Cell uptake assays were performed in cultured PC12 cells with Alexa488 and Alexa688 labeled TTC and the positive results from these studies confirmed the structural and functional integrity of the recombinant protein, post purification. Optically and nuclear labeled compound were injected into the soleus muscles of C57bl mice, and performed CT-SPECT imaging studies and biodistribution studies, which indicated nerve uptake of the intramuscularly injected compound. In vivo optical imaging of the sciatic nerve was performed with the Xenogen IVIS 200 fluorescent imager and with the Mauna Kea Cell-vizio fiberoptic system, and also demonstrated nerve uptake of the compound after intramuscular injection. The whole body pharmacokinetics of the labeled nuclear compound has been measured, and found it to be modeled by a biexponential fit with t1/2alpha:=1.115 h (75.3% contribution) and t1/2beta=95.738 h (24.7%) after intramuscular injection into the soleus muscle Cell Studies with Alexa-TTC

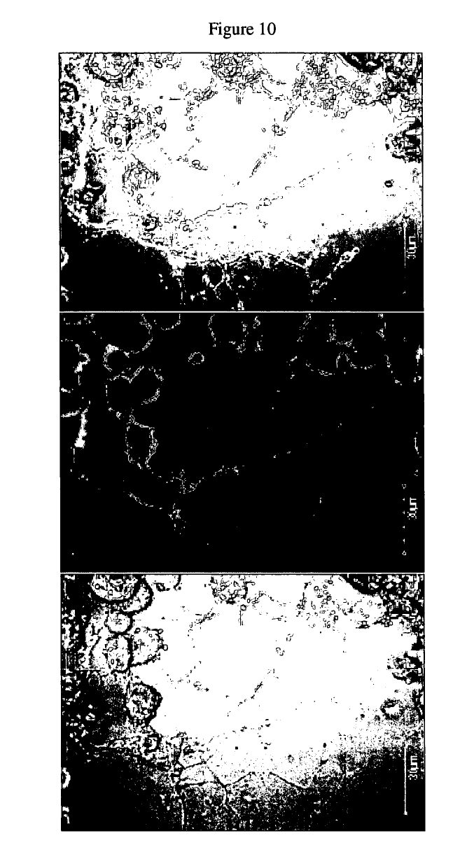

PC-12 cells (ATCC, CRL-1721), pheochromocytome cells from rat adrenal gland were cultured in DMfEM/F12 with 15% horse serum. Cells were grown on slides coated with 10% matrigel for 24 hours to 20% confluence. The cells were differentiated with 15 ng/ml NGF overnight. The cells were incubated with 4 ug Alexa-TTC/250 μl media for 4 hours. The cells were viewed using confocal microscopy, Olympus FluoviewFV 1000 (FIG. 10).

TTC Uptake and Transport

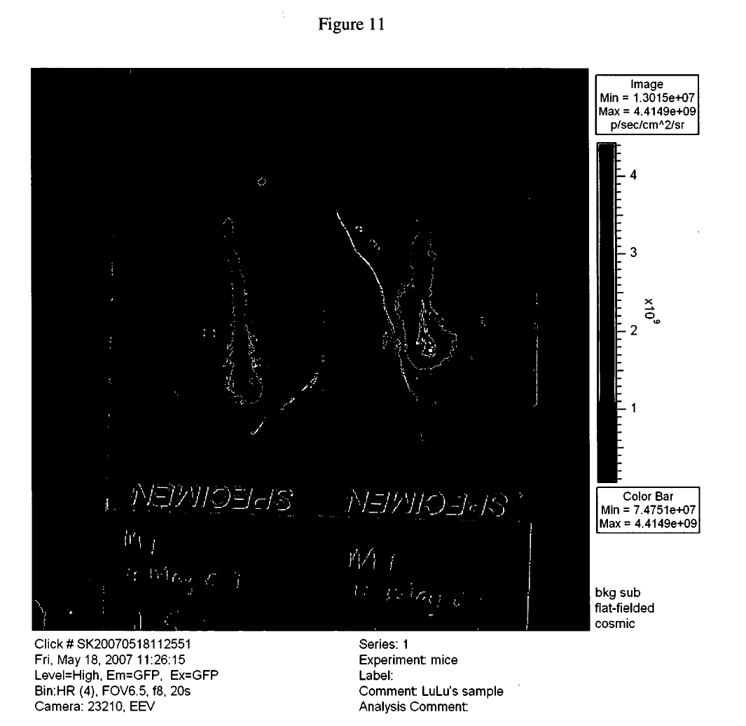

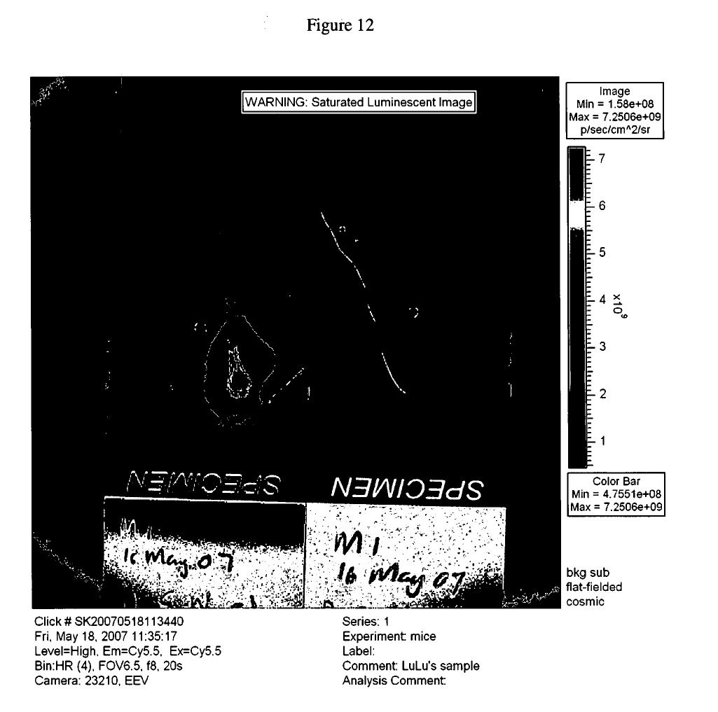

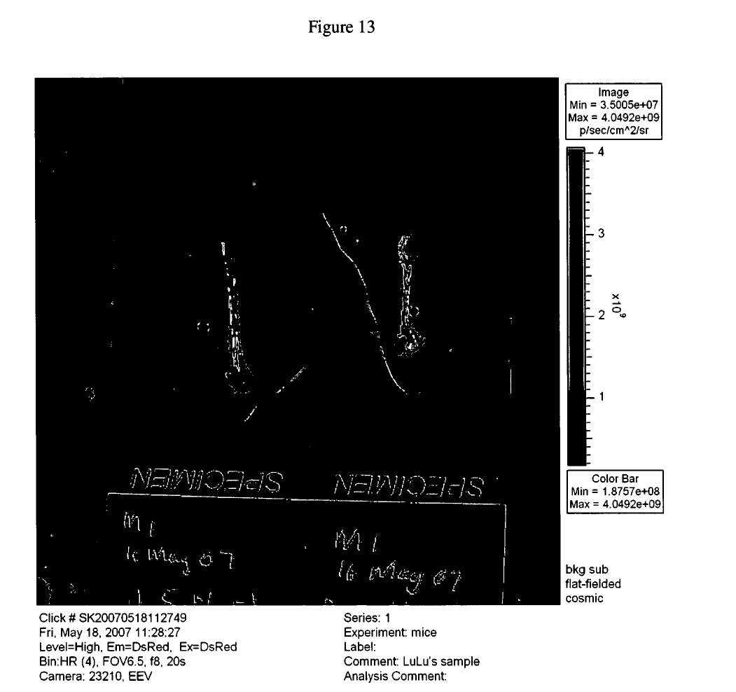

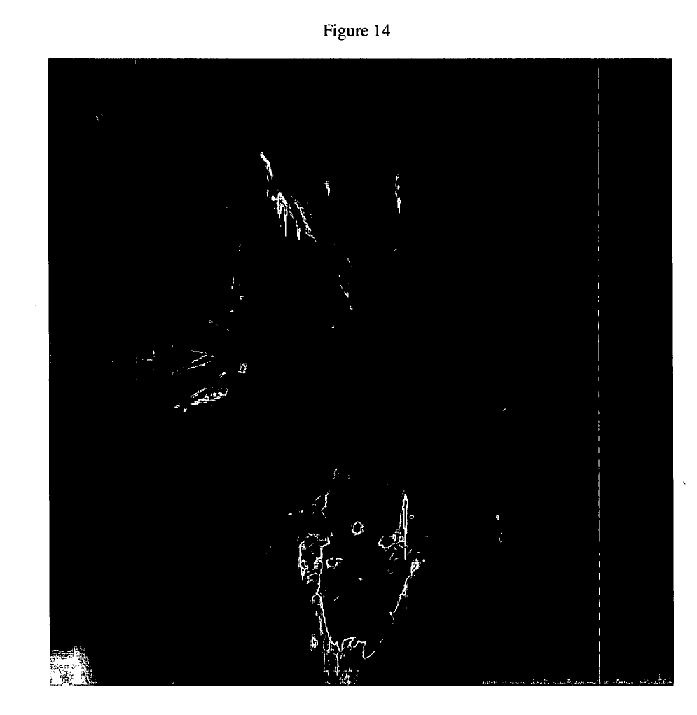

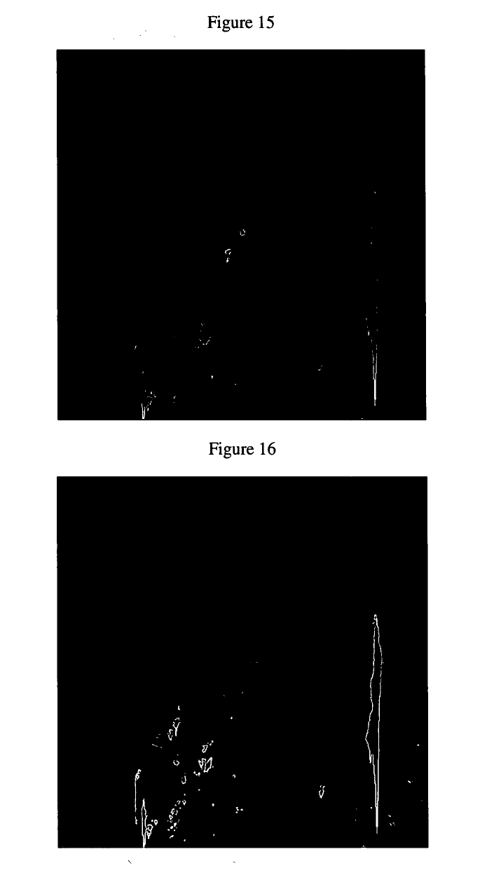

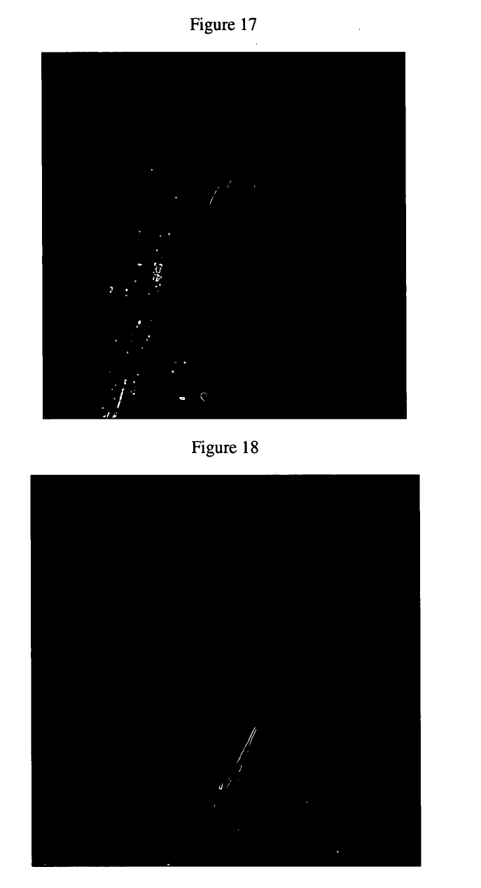

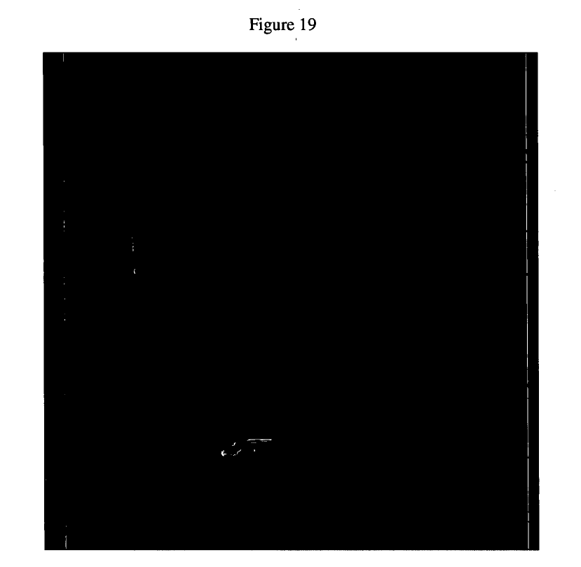

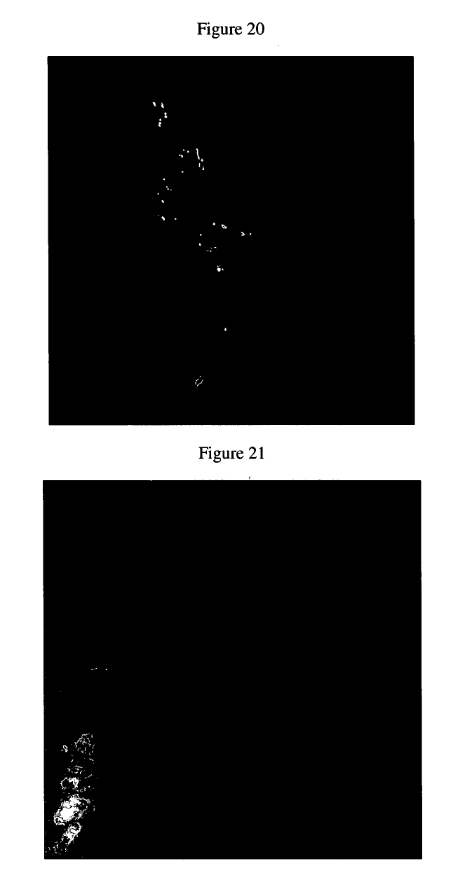

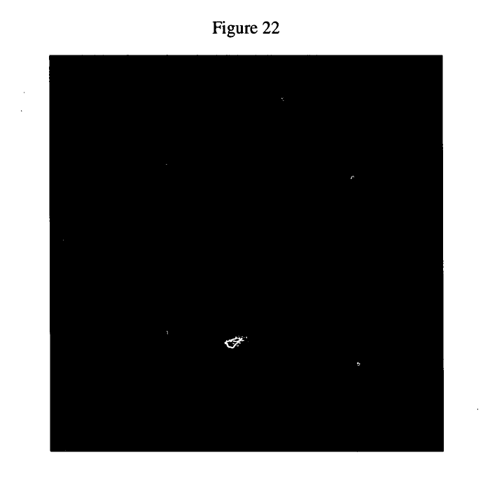

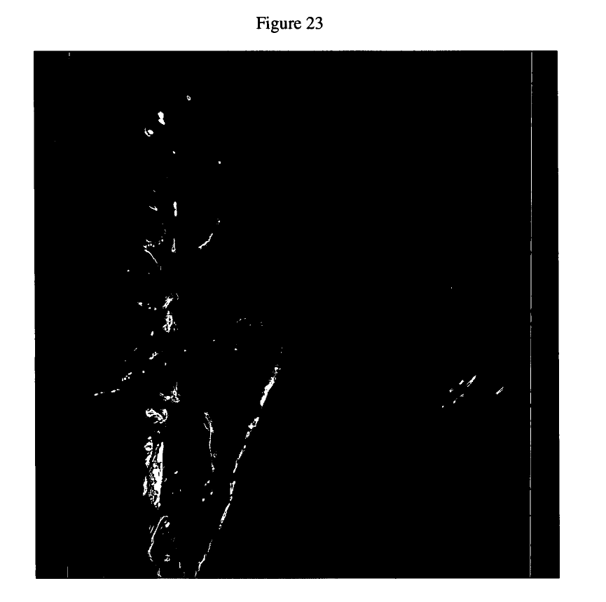

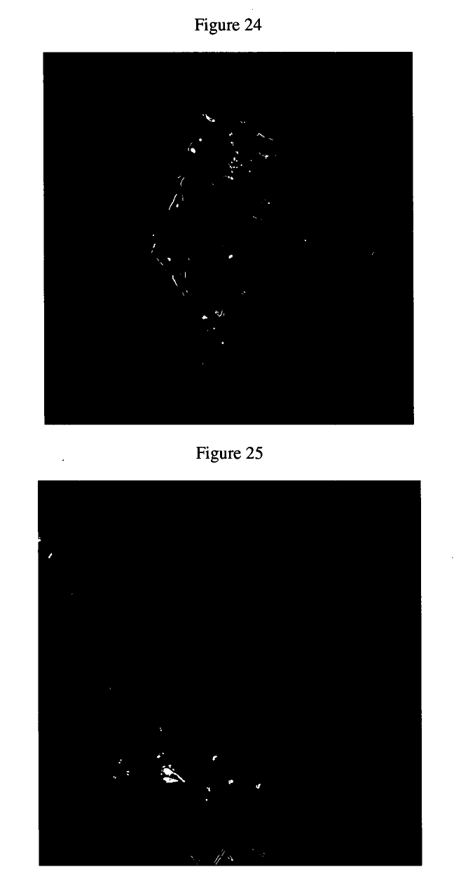

3 C57BL6 mice were injected with 80 ug/20 uL TTC-Alexa488 in the right soleus and 40 ug/20 uL HAS-Alexa680 in the left soleus and were sacrificed after 5, 24, and 36 hours. During the time between injection and sacrifice, as well as after sacrifice, one or more images of each mouse were taken with an OV100 fluorescent imager (FIG. 11-FIG. 25) to assess the time course of TTC transport in nerves. The time course of TTC transport was found to vary between specimens, and the OV100 fluorescent imager was more effective than the Xenogen fluorescent imager.

The Effect of Temperature Changes and DOTA Chelation on the Immunoreactivity of His-tagged TTC

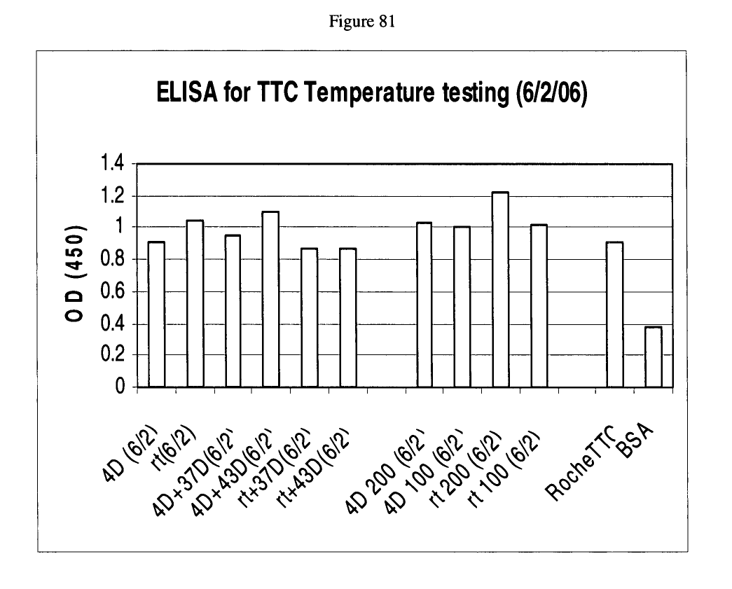

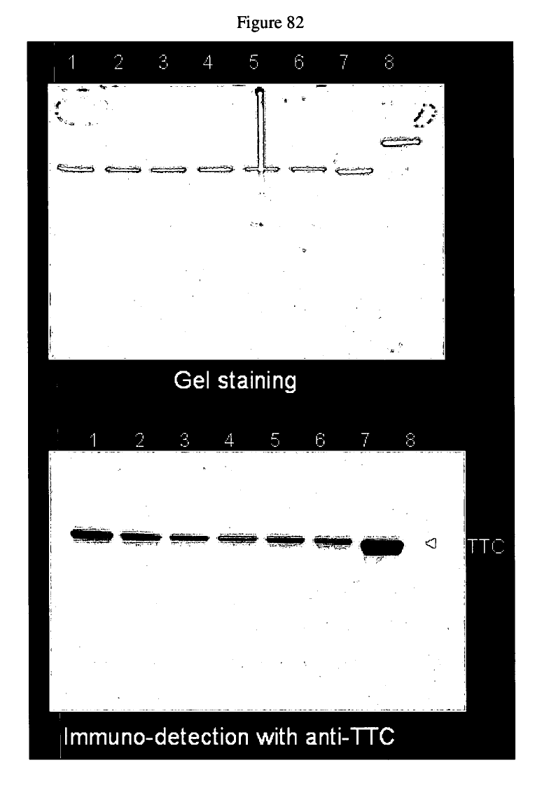

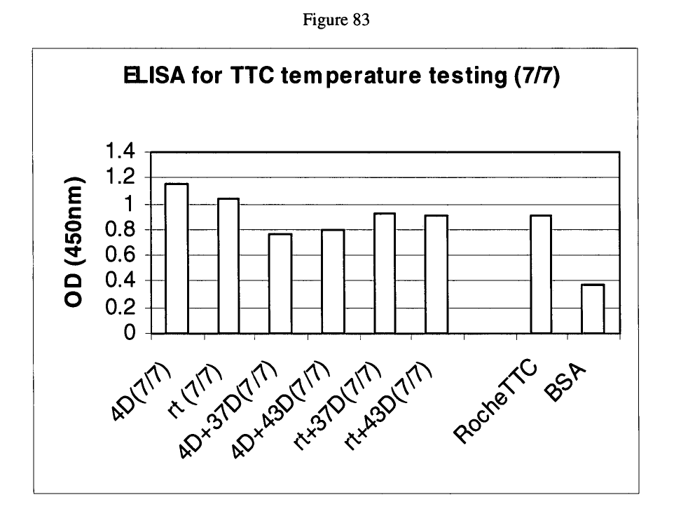

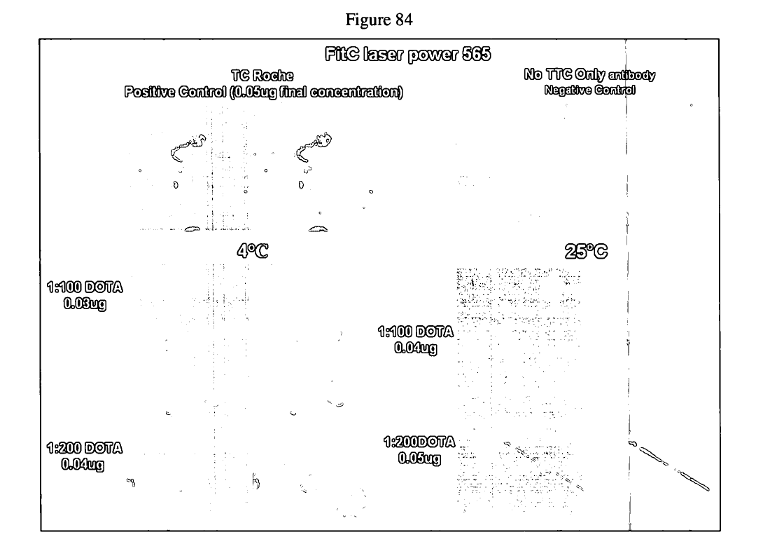

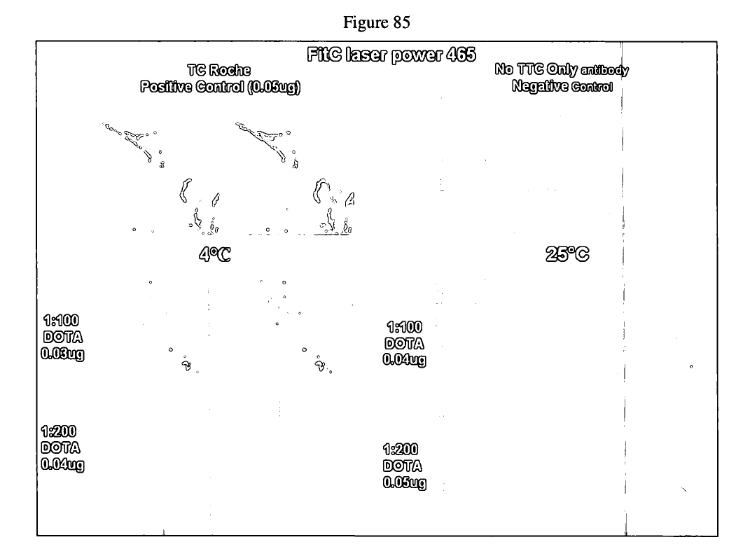

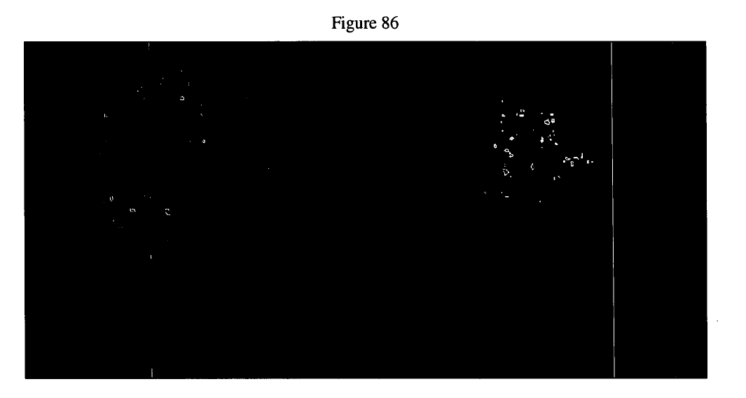

His-tagged TTC was stored during a 24-hour period under varying temperature conditions including: 4 degrees Celsius, room temperature (27 degrees Celsius), 37 degrees Celsius, 43 degrees Celsius, and combinations thereof Following the 24-hour period, the proteins were run on an SDS-PAGE gel, followed by Western blotting and immunodetection. An ELISA was also performed on the samples. This experiment was performed on two occasions, the first shown in FIG. 81 and FIG. 82, and the second in FIG. 83 and FIG. 84.

Neuronal Labeling and Immunodetection of His-TTC in PC 12 Cells

PC-12 cells were seeded at a density of 20 000 cells/well and exposed to NGF on 12 mm glass coverslips covered with poly-D-lysine (Sigma). The cells were then left to attach and form neural processes for 2.5 days. Cells began forming neural outgrowths and were at about 30% confluency when grown on poly-D-lysine coverslips. Cells on clear uncoated coverslips were attached poorly and had less neural processes. Cells on coverslips were then removed from media and excess fluid removed by Kimwipes. The cells were subsequently exposed to TTC in 0.1M Na2PO4 buffer (pH 8.5) labeled with NHS-DOTA at 4° C. or 25° C. with either 1:100 or 1:200 excess DOTA. All protein was solubilized in 20 uL droplets of PBS and PC12 cells on Coverslips were exposed to these droplets, covering all cells for 85 minutes at 37° C. in a humid cell culture incubator. After incubation, cells were washed and then fixed with 5% formalin for 5 minutes. Post-fixation, cells were washed and then exposed to an antibody regimen consisting of exposure to a primary antibody at 5 mg/ml (TC Roche Cat #1 131 621 batch 933 53220) for 1 hour followed by 3 washes and subsequent exposure to a secondary antibody 1:100 (2.5 uL: 250 uL) Zymed anti FitC (Cat# 81 65511 batch 505 94880) for 30 minutes followed by 3 washes. The cells were then mounted in Molecular Probes anti-fading medium and viewed with a Confocal FV 1000 microscope.

Animal Imaging

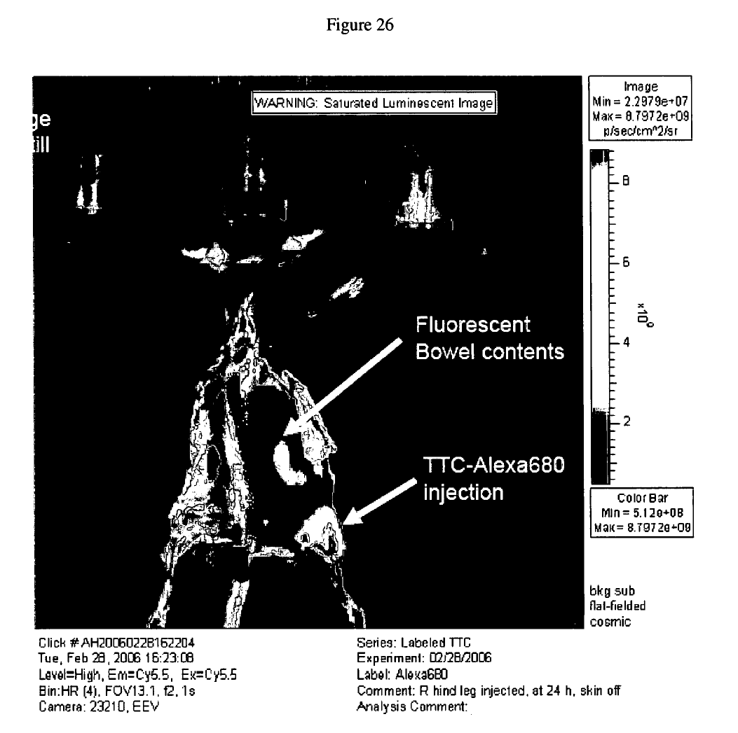

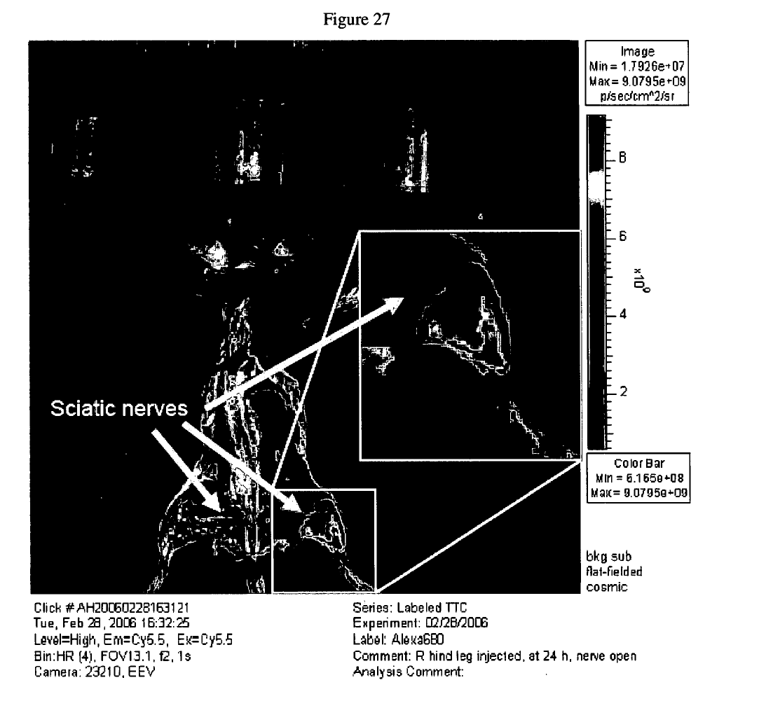

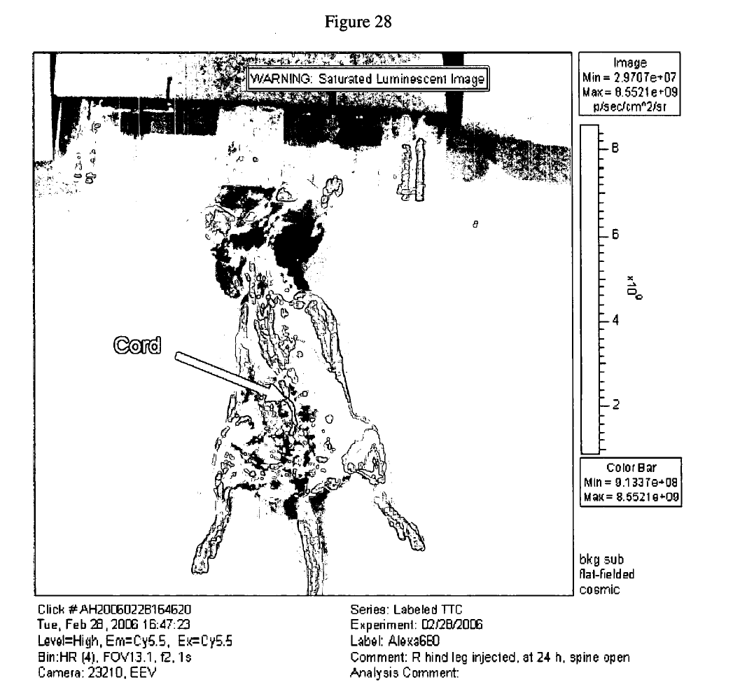

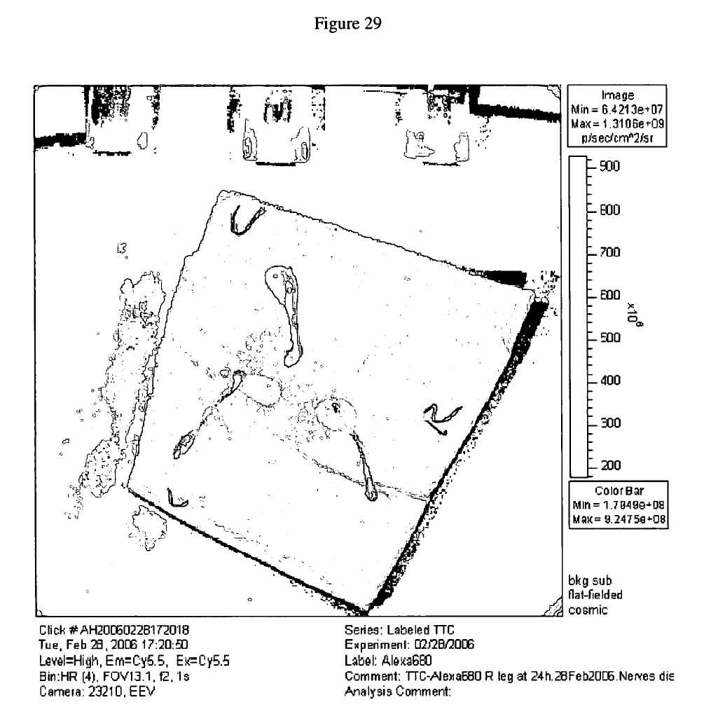

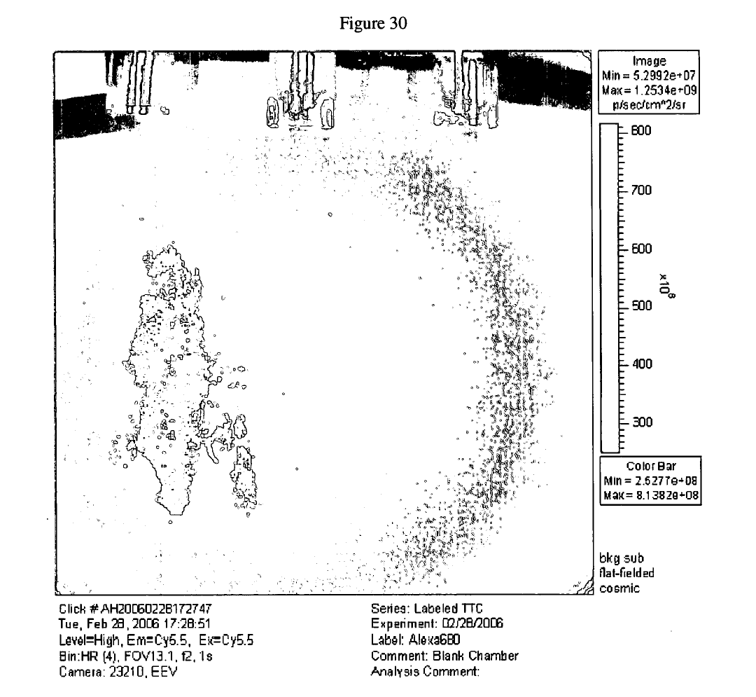

200 ug of Alexa680-TTC was injected into the gastrocnemicus muscle in 200 uL of PBS. Imaging was performed on the XenogenIVIS 200 system using the CY5.5 filter set through various phases of dissection at 24 hours after the injection (FIG. 26-FIG. 30).

Alexa680-TTC In Vivo Assay

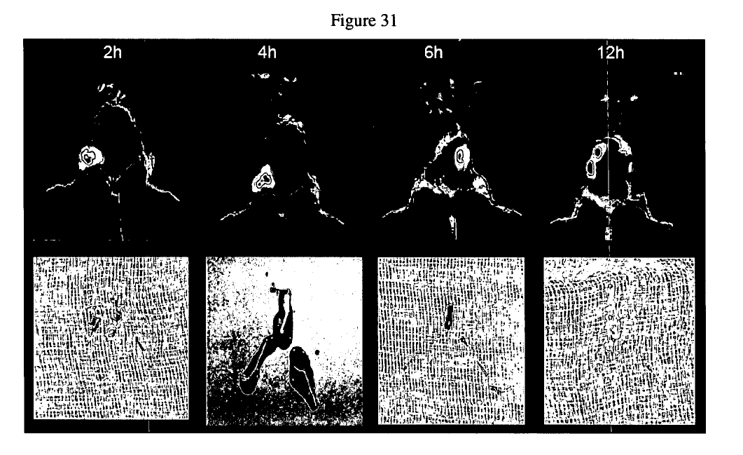

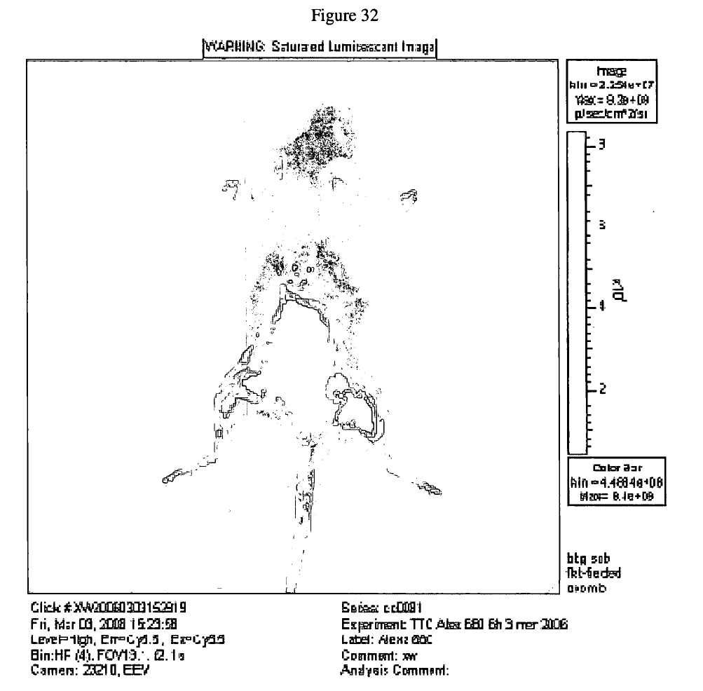

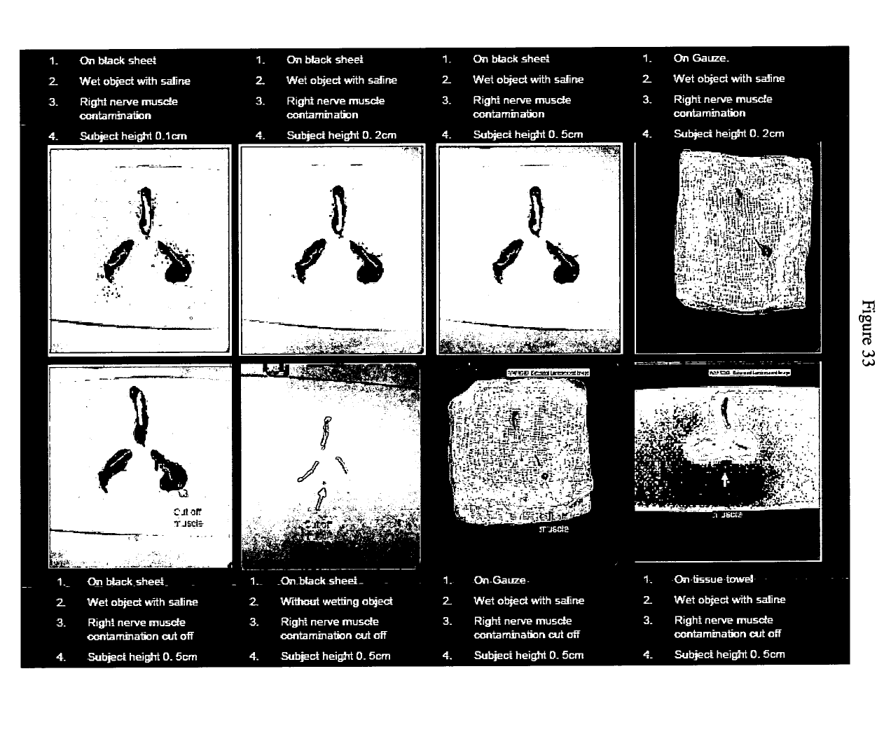

The in vivo distribution of TTC was evaluated using the Ivis200 imager over a period of 12 hours. The mouse was C57BL/6. In this in vivo time course study, Alexa680-TTC was injected into the gastrocnemicus (50 ug/50 uL) in C57BL/6 mice (FIG. 31) White cotton appears to be a better background than black matte paper for imaging excised organs (See FIG. 31). Alexa680-TTC in vivo assay was repeated for examination of Alexa680-TTC distribution after 6 h of treatment using the same type of mouse and dose of Alexa680-TTC. The mouse was injected with Alexa680-TTC through right sciatic nerve. Imaging was performed using an Ivis 200 imager on the whole mouse (FIG. 32) and on excised organs (FIG. 33). TTC is taken up into nerves, and using ex vivo fluorescent imaging, it can be seen that gauze is the best background for excised organs.

TTC-His Conjugation with EC

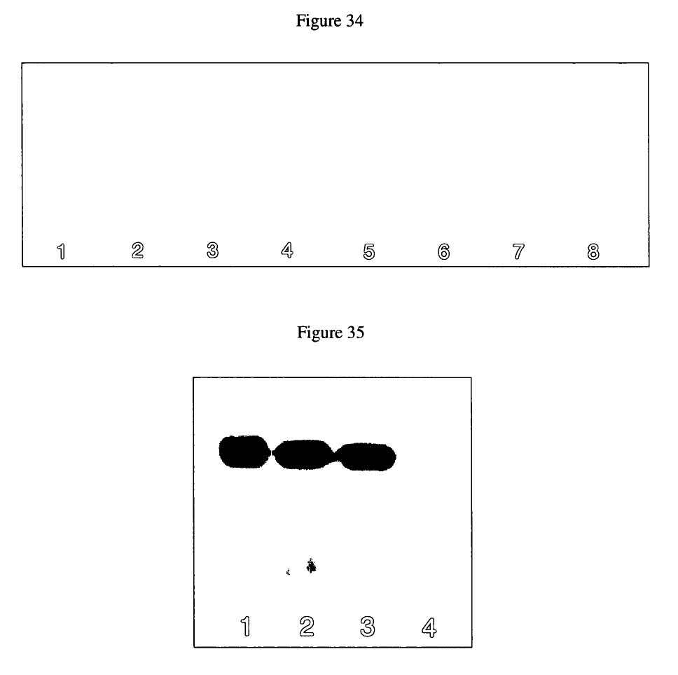

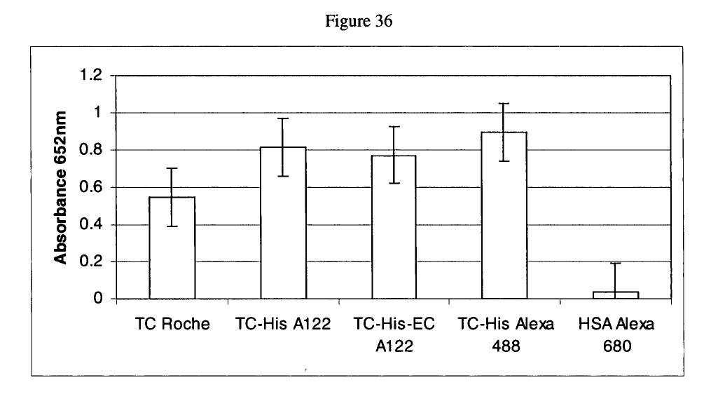

0.15 mg ethylenedicysteine (EC), 0.12 mg N-hydroxysulfosuccinimide (Sulfo-NHS), and 0.107 mg 1-(3-Dimethylaminopropyl)-3-ethylcarbodiimide hydrochloride (EDC) were added to 1 mL of 1.5 mg/ml TTC-His. Sulfo-NHS and EDC are the catalysts for the conjugation. The mixture was permitted to react overnight at room temperature. The protein was then dialysed (MW<10.000) for 8 hours, changing the dialysate every hour. The product was then freeze dried. Following the conjugation procedure, the sample underwent SDS-PAGE, Western blotting and immunodetection, and an ELISA assay with appropriate controls, as shown in FIG. 34-FIG. 36.

PC12 Uptake Studies

Uptake studies on PC12 cells were performed with the various TC conjugates, PC12 cells were seeded in 96-well flat-bottom plates at a density of 2,000 cells/well. After 24 hours, 50 ng/ml NGF was added to the media. Media was then changed at 2 and 5 days after seeding, and the uptake study was performed 8 days after seeding.

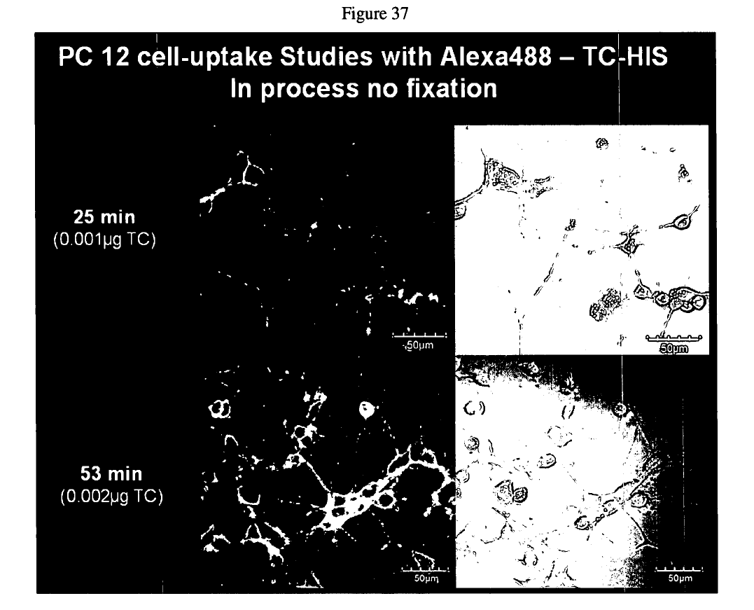

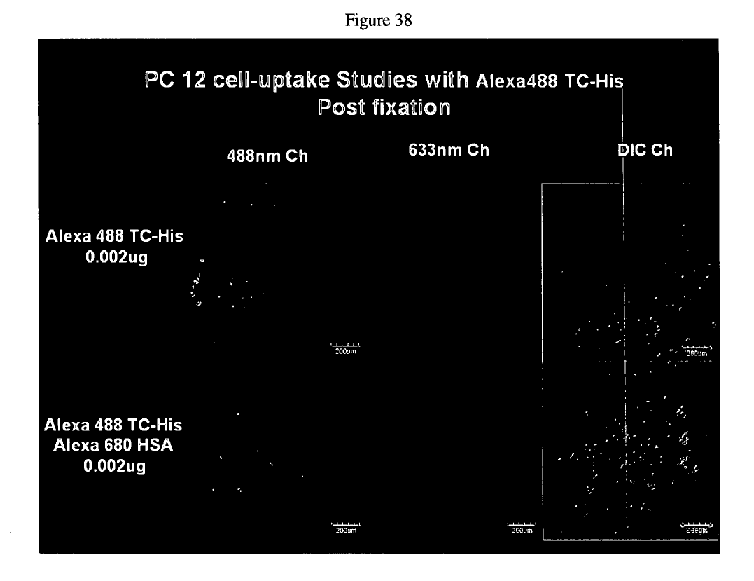

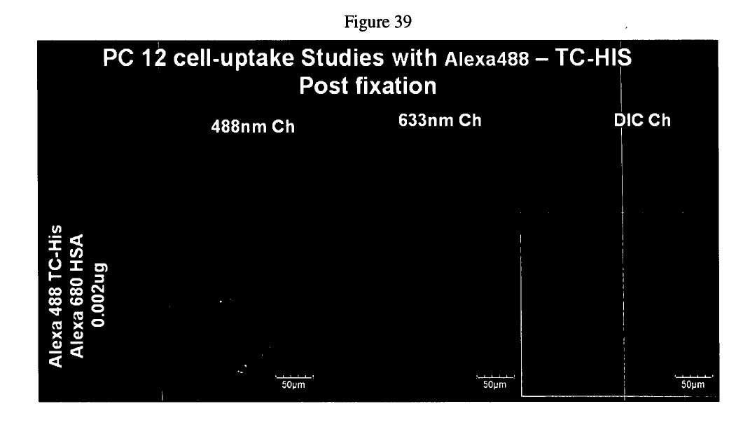

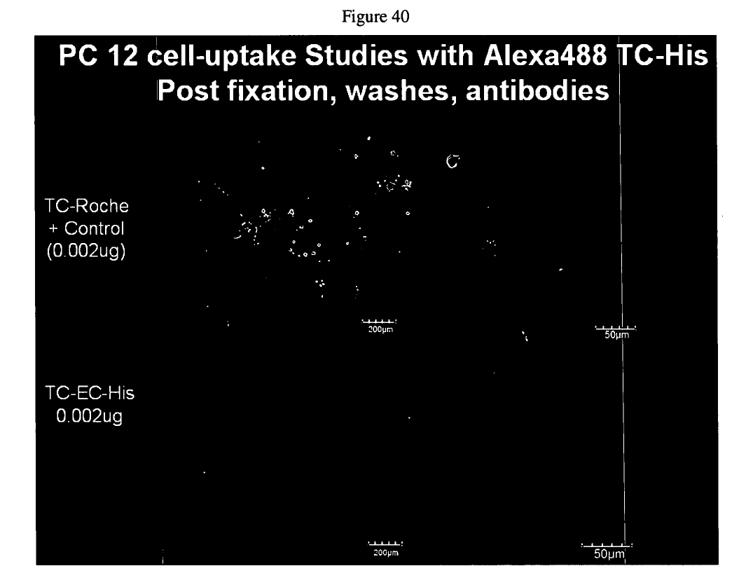

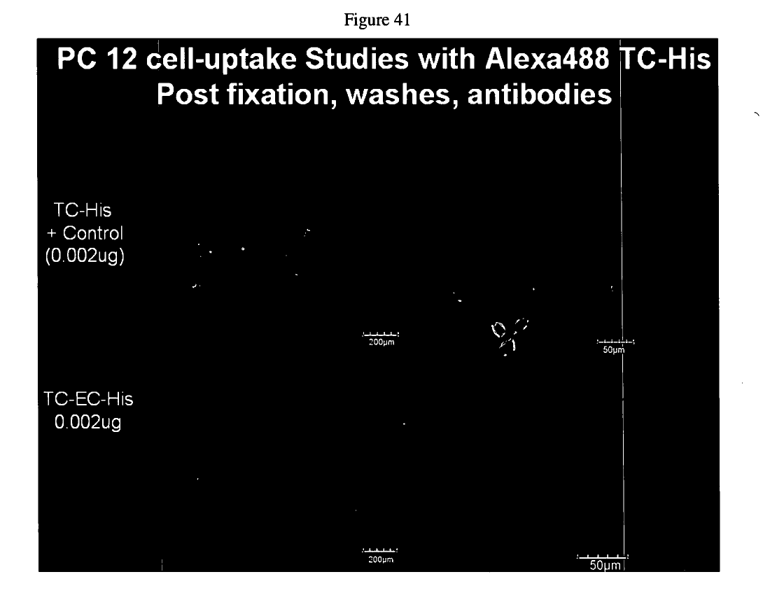

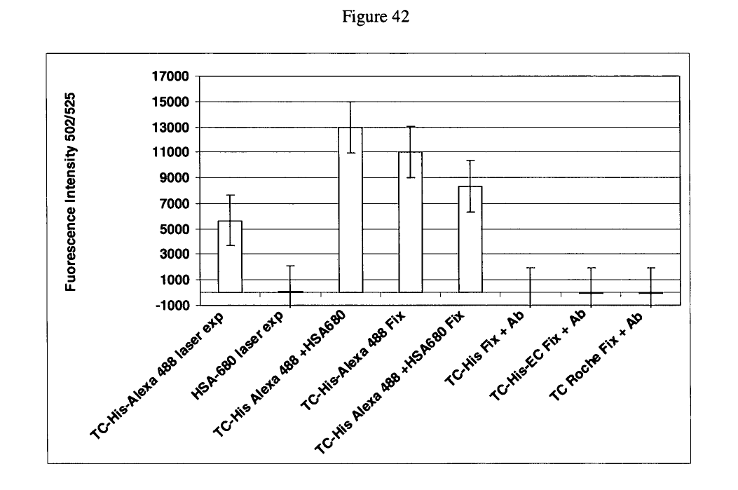

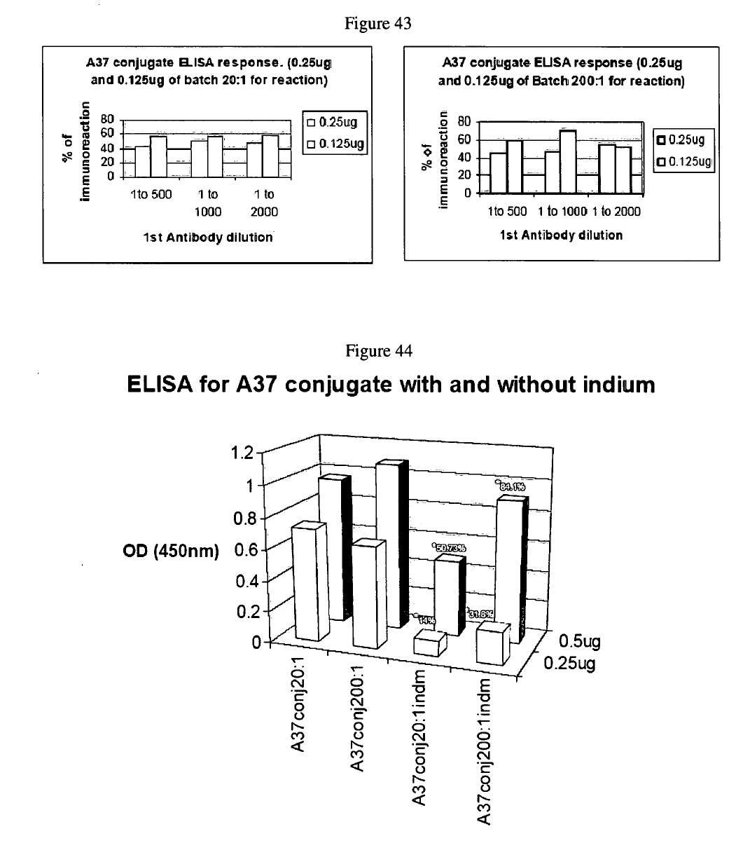

The uptake study was a multi-step process. First, 1-3 ug TC-Alexa488, 1-3 ug HSA-Alexa 860 and combinations of both were added to cells. Uptake of the conjugates was observed under a confocal microscope at 37° C. for 1 hour. A second step involved repeating the above step, followed by fixation of the cells after 1 hour with 5% paraformaldehyde at room temperature for 5 min. Cells were then washed and observed under a confocal microscope. A positive control (TC-Roche) was used in this experiment. The cells were first exposed to a primary anti-TC monoclonal antibody (diluted 1:2000) for 1 hour and then to a secondary anti-FITC anti-body (diluted 1:2000) for 30 minutes. Following two washes with 0.5% BSA in PBS, the cells were observed with an FV1000 confocal microscope (FIG. 37-FIG. 41). An ELISA was also performed on the cells (FIG. 42).

TTC-DOTA-Indium Labeling and Conjugating TTC to NHS-DOTA

TTC was dialyzed overnight to 1 L Tris 0.3 M, pH=8, with Chelex 100 1.2 g. The TTC was incubated with NHS-DOTA at molar excess of 20, 100, and 200 at 25 ° C. for 24 h with end over end mixing. The protein was dialyzed again to 1 L Tris 0.3 M, pH=8 and Chelex. Indium-trichloride was prepared with ammonium acetate and citric acid to a weak citrate-acetate chelate. This weakly chelated Indium was incubated with TTC-DOTA which then transchelates the Indium to DOTA. Immunoreactivity of the conjugate (A37) was assessed from ELISA assay (FIG. 34). % of immunoreaction is % of control calculated as OD value of A37 conjugate versus OD value of A37. It reflects only the ability of protein recognizable by its specific antibody. It does not provide any information about the binding efficiency of the conjugate.

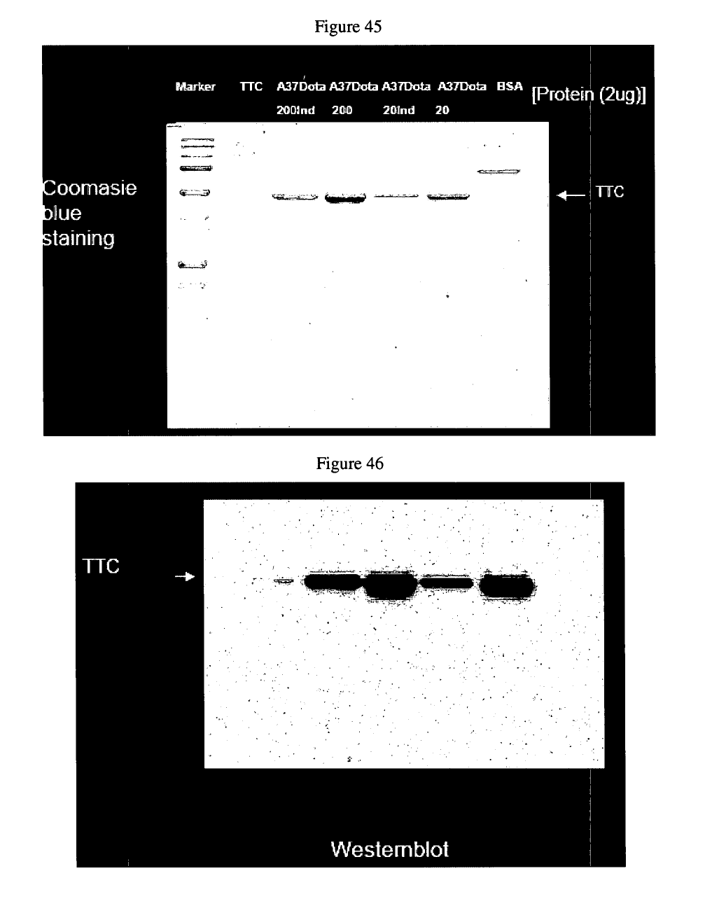

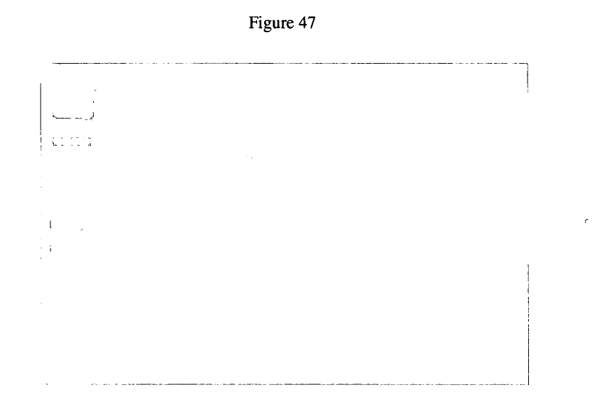

For the number of chelex per protein molecules, have an iTLCassay is still needed. The results indicate that 1 ug of conjugate gives % of immunoreaction (% of control) at around 98%, although the OD value of 1 ug TTC showed that it is out of scale. The 0.25 ug and the 0.125 ug gives close to consisitant results. (FIG. 37). According to this figure (0.25 ug), the overall % of immunoreaction (% of control) is around 50% average for both batches. Although 0.125 ug gives relatively higher immunoreactivity percentage, its OD value seems lower that common acceptable value (>0.2). So 0.25 ug or 0.5 ug should be a good amount for this response. First antibody could be diluted 1:2000 according to FIG. 44. FIG. 44 shows ELISA assay results for conjugate with and without indium. FIG. 45-FIG. 47 shows gel staining and western blot of TTC labeling with DOTA Chelator.

Indium-111 Labeling of TTC-DOTA

600 uL of 0.3 M ammonium acetate at pH 9 was mixed with 400 uL In-111-trichloride in 0.05 HCl at pH 1-1.4. After 10-15 minutes, 250 uL of “In-Acetate” solution was transpipetted to each of 4 protein-DOTA conjugates. DOTA20, DOTA100, DOTA200A and DOTA200B. The samples were allowed to incubate overnight at room temperature. Table 1 below shows the TTC-DOTA-Indium labeling. This indicated that very poor labeling was achieved. Heating at 43° C. for 1 hour did not improve the results.

TABLE 1

| TTC-DOTA | Pure (%) | Retained | [Protein mg/mL] |
|---|---|---|---|
| DOTA20 | 12 (5%) | 220 uCi | 0.12 |
| DOTA100 | 8 (5%) | 141 uCi | 0.34 |
| DOTA200A | 8 (3%) | 260 uCi | 0.12 |
| DOTA200B | 9 (6%) | 141 uCi | 0.34 |

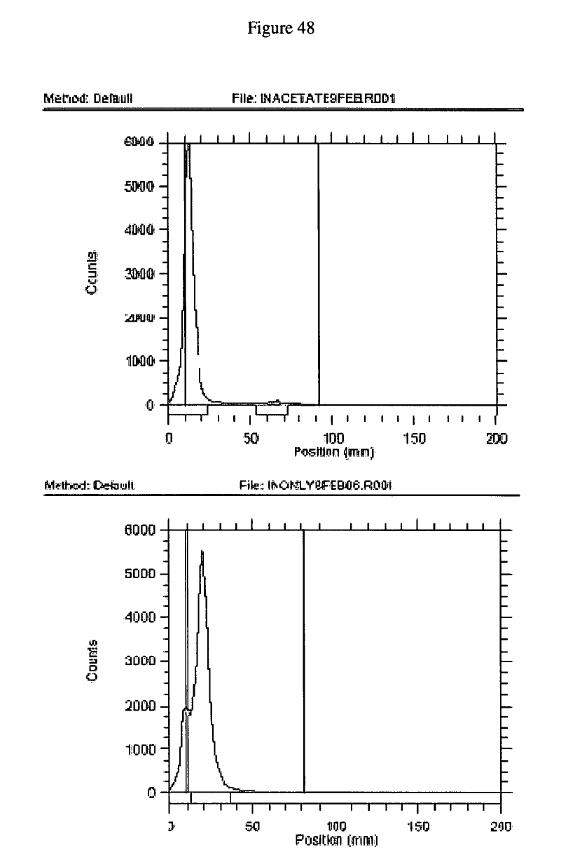

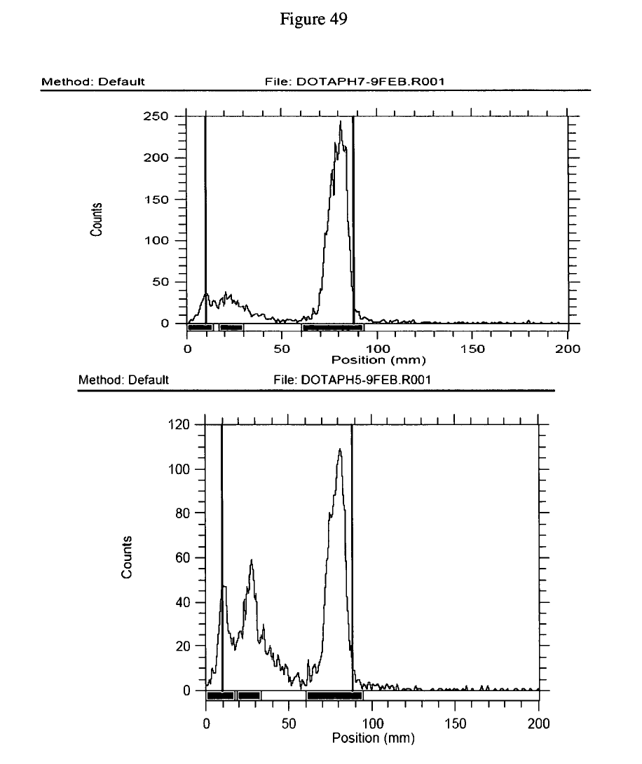

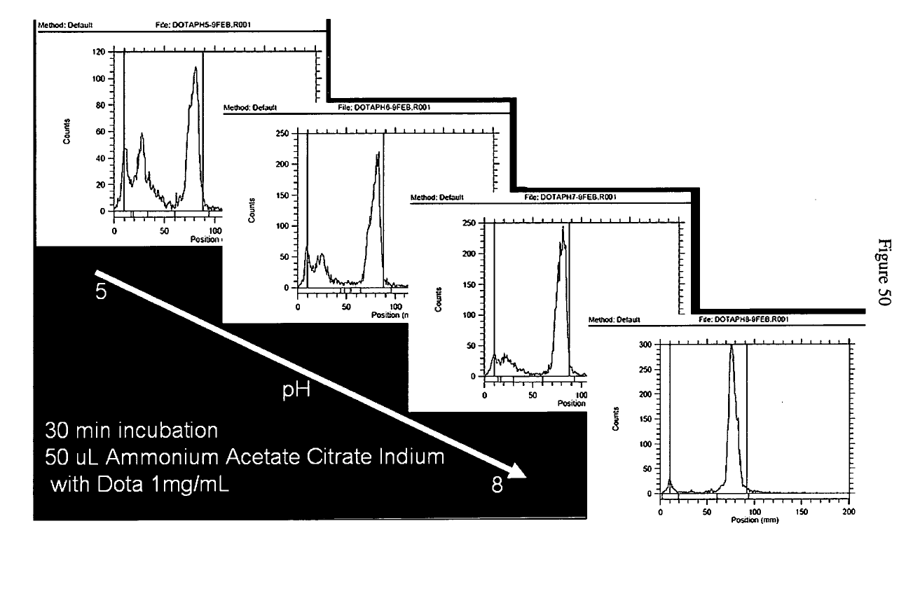

Thin layer chromatography (TLC) was performed on the samples. 80:20 MetOH:Water on Cellulose does not appear to separate ionic Indium-T111 and Indium-Acetate. TLC cannot be used to assess labeling in its present form (FIG. 48). Saline TLC on Cellulose discriminates between ionic Indium and weak citrate-acetate chelates of Indium. Conditions need to be optimized for the formation of weakly chelated species. (FIG. 49). DOTA-Indium chelation showed a pH dependence (FIG. 50) For pH of about 5, 6, 7, and 8, Indium chelation was 59%, 66%, 83%, and 95%. Higher pH enhances DOTA chelation.

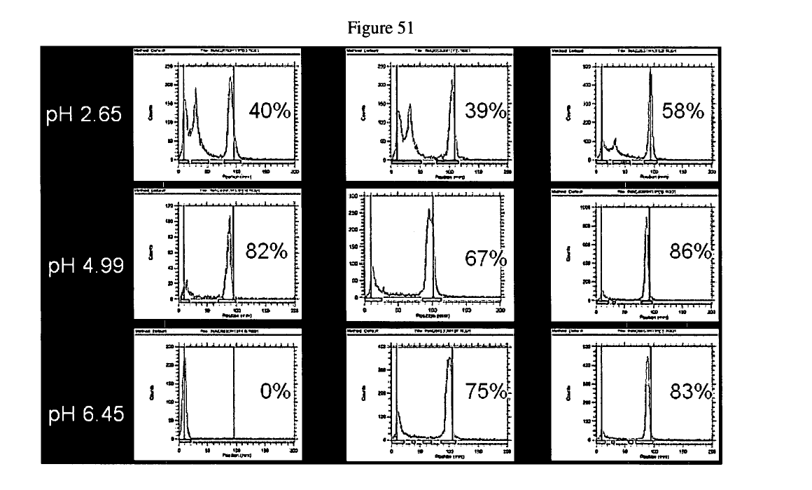

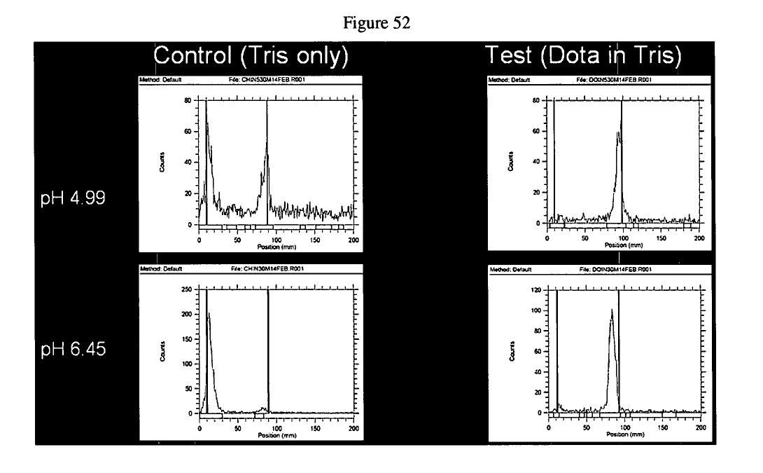

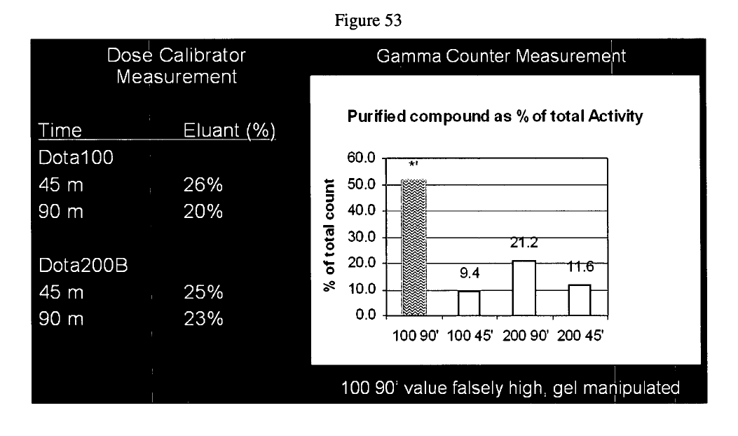

Optimization of Indium-Acetate (Citrate) weakly chelated species in solution was assessed using TLC with respect to pH and time. (FIG. 51). Cellulose-Saline TLC was performed after incubation of 40 uL In-111-trichloride in 0.05 HCl (pH 1-1.4), 100 uL ammonium acetate (0.1 M, pH 7.2), 250 uL citric acid (0.1 M, pH varies 1.7, about 4, and about 7) for final pH as shows in FIG. 51. Optimization of Indium-Acetate binding to DOTA was assessed with respect to pH (FIG. 52). Cellulose-saline TLC was performed after 30 minute incubation. InAc at stated pH in figure was combined with 200 uL Tris (pH 8) with or without DOTA. Binding of TTC-DOTA to In-Acetate was also assessed at a pH 5 preparation (FIG. 53).

Animal Studies

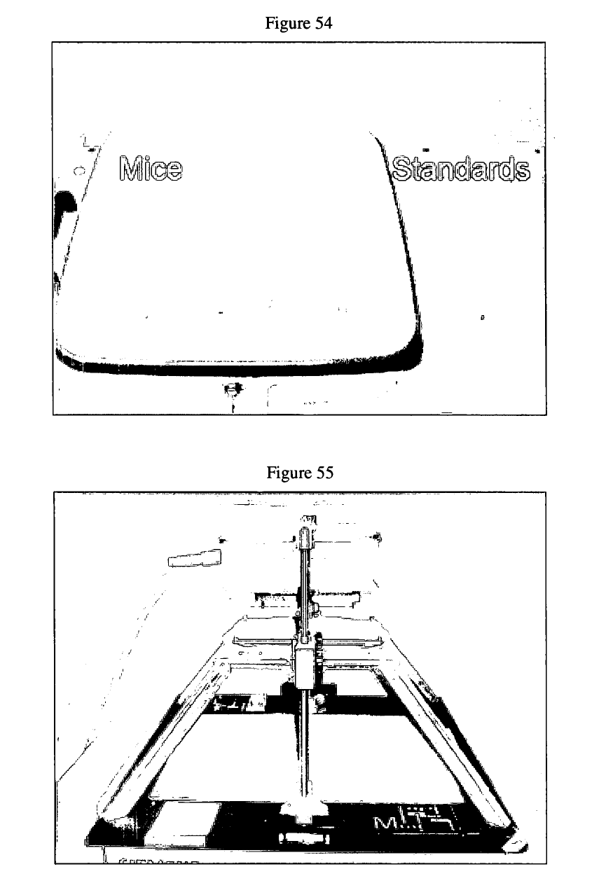

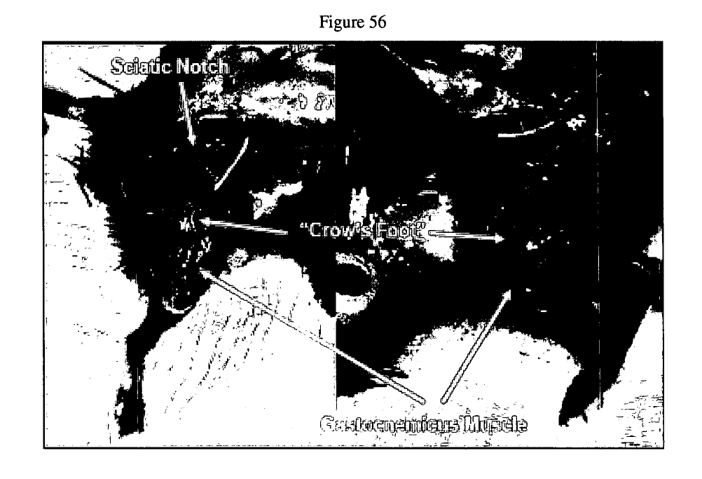

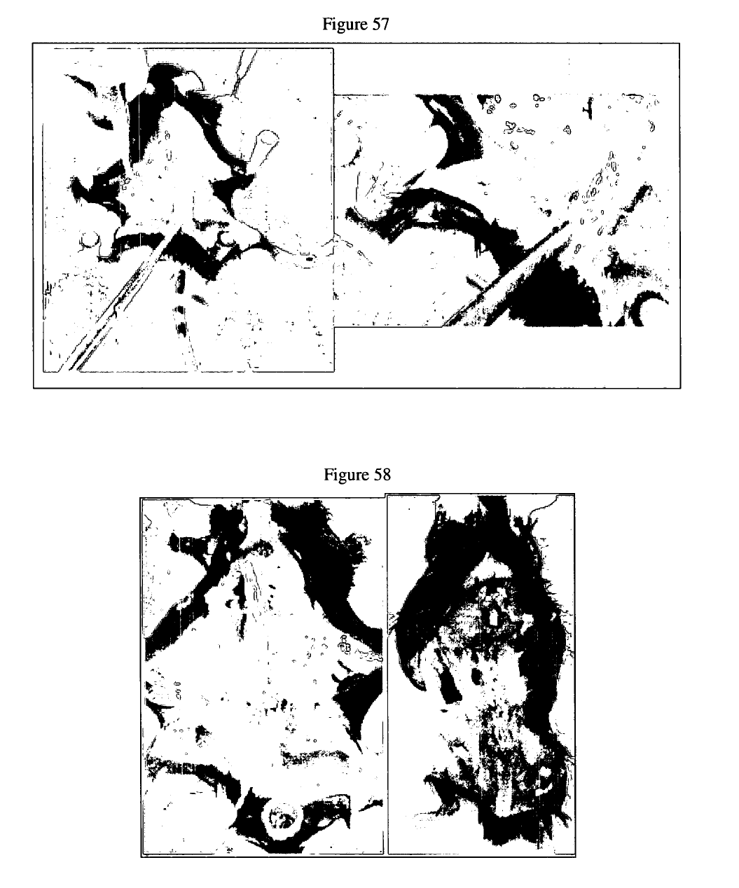

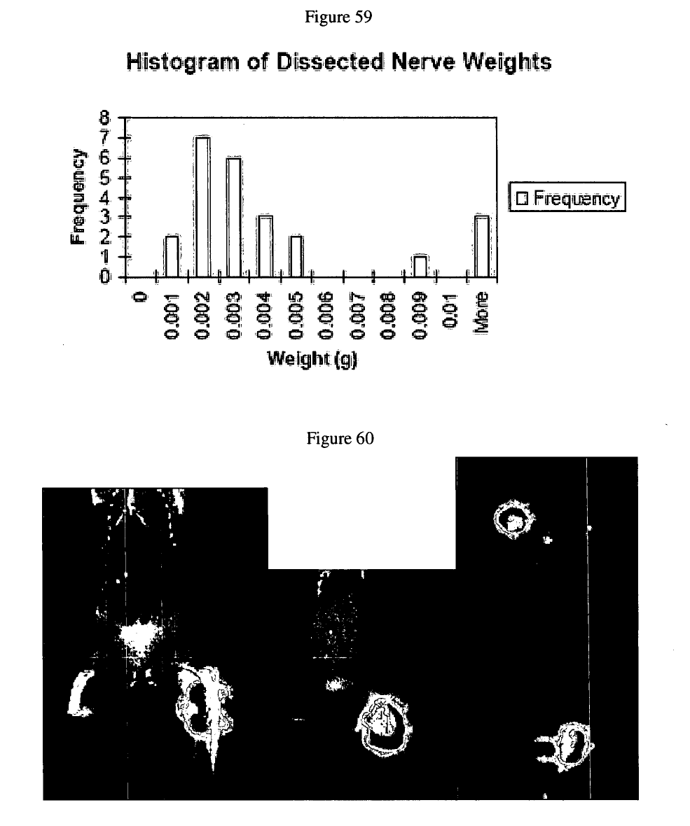

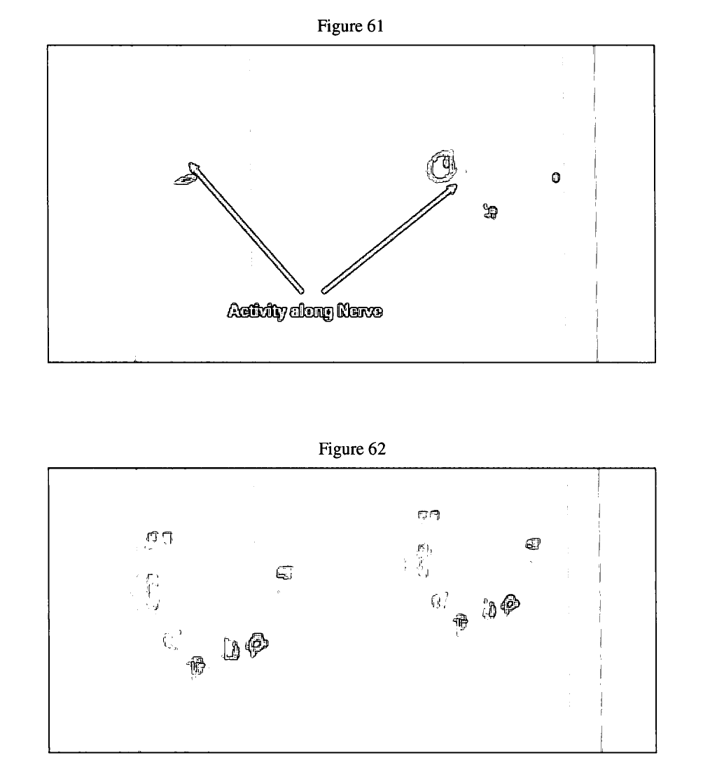

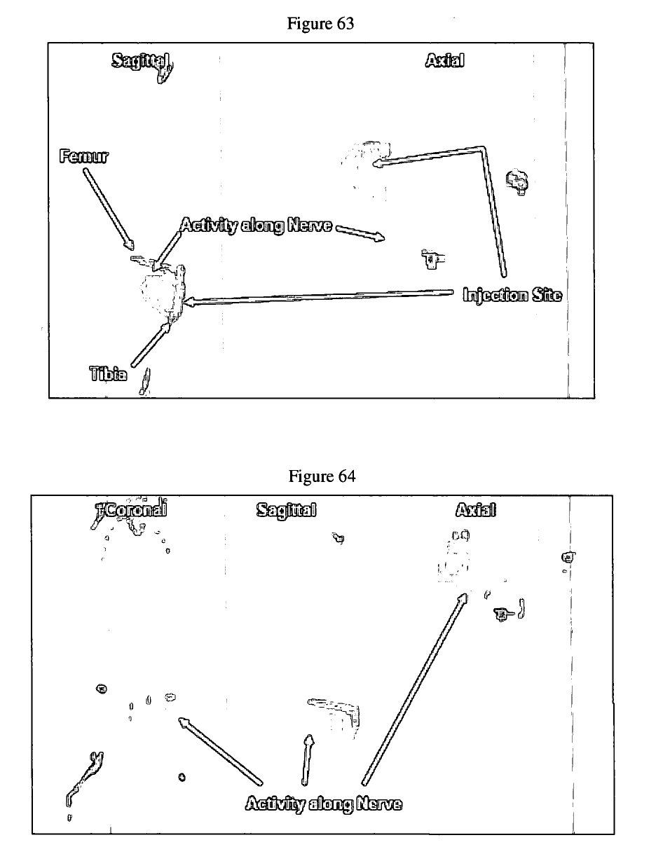

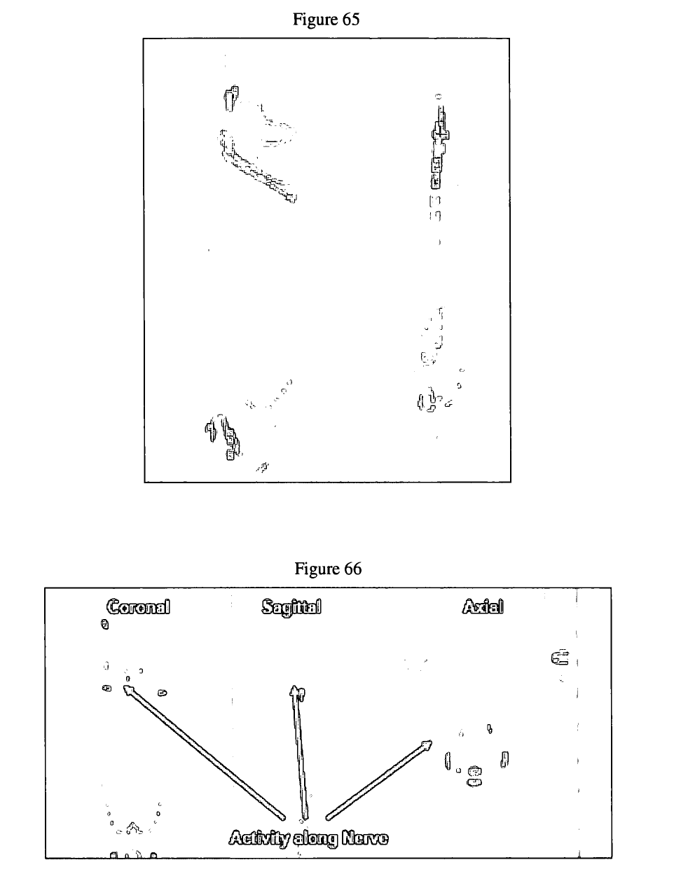

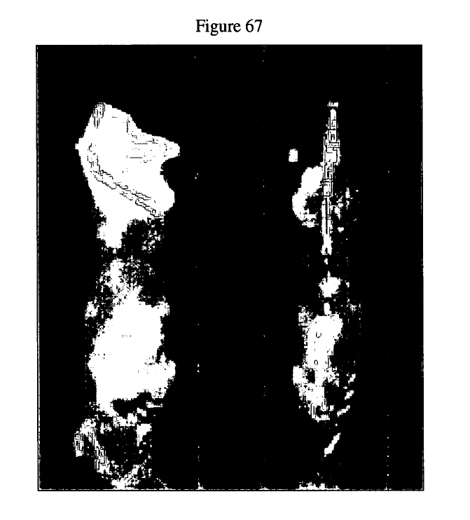

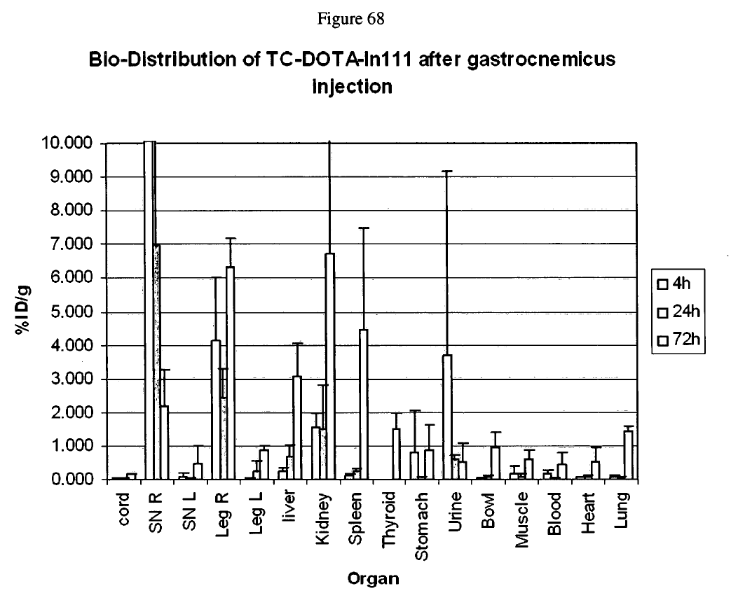

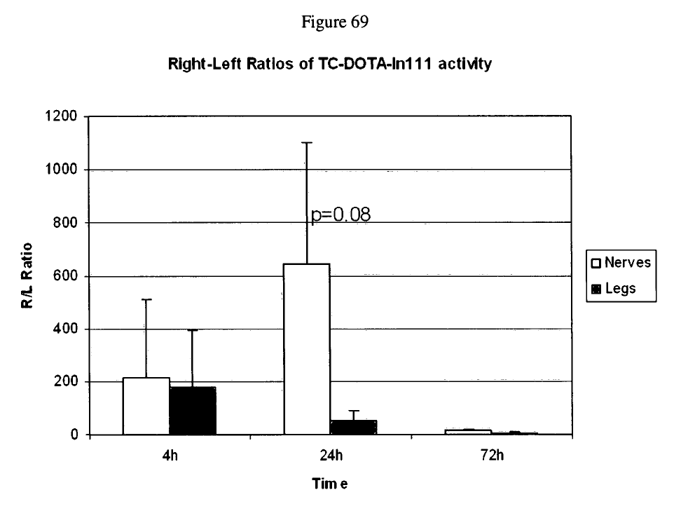

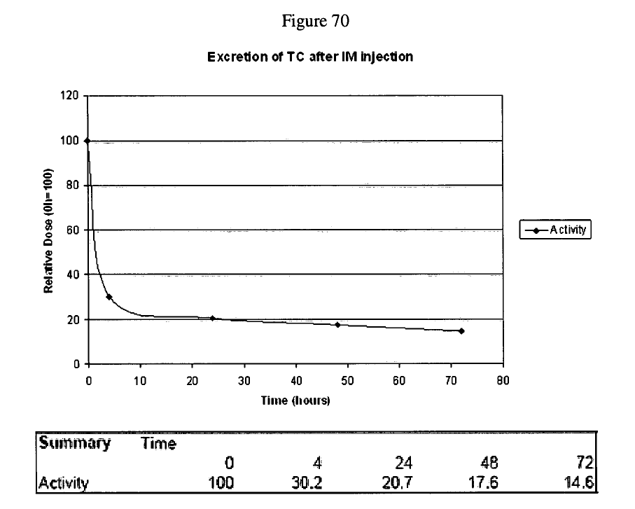

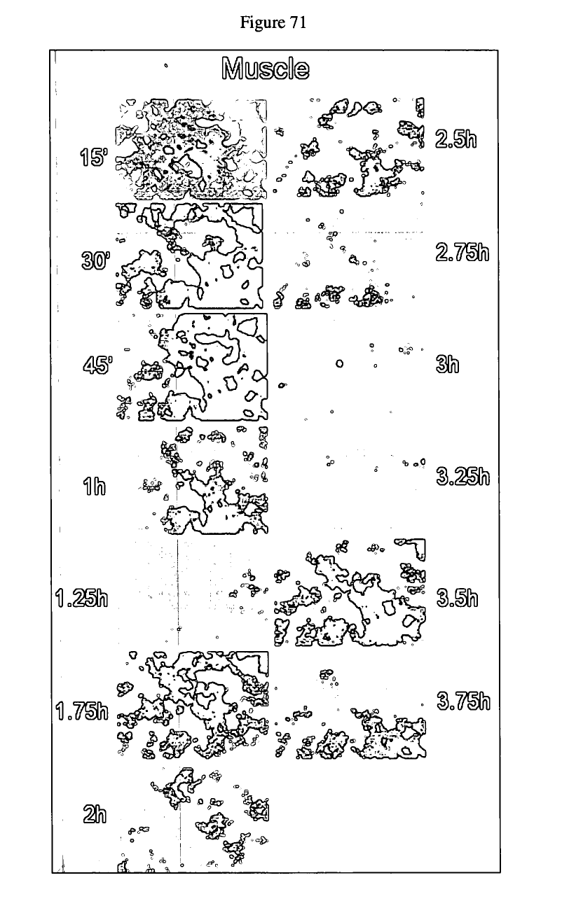

MCAM imaging procedure and coded aperture was used as shown in FIG. 54 and FIG. 55. Dissection Procedure images are shown in FIG. 56-FIG. 58. FIG. 59 shows a histogram of dissected nerve weights. The results show that there is too much variability among samples, and dissection needs to be standardized. Biodistribution studies were performed after gastrocnecimcus injection of TC-DOTA-In 111 Table 2, 3, and 4 below show the results of the study. Table 2 shows the distribution with the mouse being sacrificed 4 hours after injection. Table 3 shows the distribution after sacrifice of the mouse 24 hours after injection. Table 4, show biodistribution after sacrifice of the mouse 72 hours after injection. The mice were imaged at an 0 hours, 8 hours, 24 hours, 27 hours, 28 hours, 30 hours, and 48 hours (FIG. 60-FIG. 67). There is some evidence of activity tracking along the sciatic nerve. Higher resolution imaging, which would increase specific activity, calibrate with indium, pinhole, is needed. Better sampling of early time points dynamically (CellViso, Xspect, AR) may be needed. Better injects and background decrease may also be needed. Table 5 shows a summary of the biodistribution data. FIG. 68 and FIG. 69 show biodistribution of TC-DOTA-In111 after gastronemicus injection as a function of % ID/gram after 4, 24, and 72 hours. Table 6 below shows ratio analysis across the four mice samples between the nerves and the legs at 4, 24, and 72 hours. FIG. 70 shows right-left rations of TTC-DOTA-In111 activity at 4, 24, and 72 hours in the nerves and in the legs. FIG. 71 shows an excretion profile of TTC-DOTA-In111, with a T1/2 alpha of 1.115 hours (75.3% contribution), a T1/2 beta of 95.738 hours, (24.7% contribution using a two compartment, Winonlin software. Overall, TC-DOTA -In111 accumulates in nerve tissue. Most interactions occur early, hours to a day, and excretion is renal.

TABLE 2
4 h post injection
calculated Total dose = 121573440

| | % of total dose/gm | | | | | | |
|---|---|---|---|---|---|---|---|
| Organ | mouse1 | mouse2 | mouse3 | mouse4 | mean | SD | Mean R/L |
| cord | 0.032 | 0.003 | 0.015 | 0.049 | 0.017 | 0.014 | |
| SN R | 13.580 | 0.707 | 28.382 | 3.086 | 14.223 | 13.849 | 161.541 |
| SN L | 0.021 | 0.035 | 0.208 | 0.062 | 0.088 | 0.104 | |
| Leg R | 6.291 | 2.970 | 3.145 | 5.330 | 4.135 | 1.869 | 116.195 |
| Leg L | 0.013 | 0.051 | 0.043 | 0.058 | 0.036 | 0.020 | |
| liver | 0.162 | 0.235 | 0.360 | 0.583 | 0.252 | 0.100 | |
| Kidney | 1.167 | 1.464 | 2.026 | 2.726 | 1.552 | 0.437 | |
| Spleen | 0.143 | 0.155 | 0.102 | 0.891 | 0.133 | 0.028 | |
| Thyroid | 0.050 | 0.000 | 0.000 | 0.069 | 0.017 | | |
| Stomach | 0.034 | 0.038 | 2.280 | 0.523 | 0.784 | 1.296 | |
| Urine | 0.000 | 9.968 | 1.202 | 7.110 | 3.723 | 5.441 | |
| Bowl | 0.009 | 0.034 | 0.022 | 0.310 | 0.022 | 0.012 | |
| Muscle | 0.056 | 0.023 | 0.417 | 0.043 | 0.165 | 0.218 | |
| Blood | 0.286 | 0.090 | 0.146 | 0.218 | 0.174 | 0.100 | |
| Heart | 0.071 | 0.052 | 0.074 | 0.111 | 0.066 | 0.012 | |
| Lung | 0.110 | 0.066 | 0.069 | 0.126 | 0.082 | 0.025 | |

TABLE 3
24 h post injection
calculated Total dose = 121573440

| | % of total dose/gm | | | | | | |
|---|---|---|---|---|---|---|---|
| Organ | mouse1 | mouse2 | mouse3 | mouse4 | mean | SD | Mean R/L |
| cord | 0.024 | 0.010 | 0.008 | 0.010 | 0.014 | 0.009 | |
| SN R | 11.957 | 2.881 | 6.080 | 13.805 | 6.972 | 4.603 | 363.529 |
| SN L | 0.010 | 0.007 | 0.040 | 0.016 | 0.019 | 0.018 | |
| Leg R | 1.863 | 1.946 | 3.455 | 3.431 | 2.421 | 0.896 | 10.576 |
| Leg L | 0.059 | 0.588 | 0.039 | 0.043 | 0.229 | 0.311 | |
| liver | 1.110 | 0.501 | 0.431 | 0.516 | 0.681 | 0.373 | |
| Kidney | 2.031 | 0.026 | 2.470 | 3.133 | 1.509 | 1.303 | |
| Spleen | 0.346 | 0.190 | 0.201 | 0.318 | 0.246 | 0.087 | |
| Thyroid | 0.000 | 0.000 | 0.043 | 0.000 | 0.014 | | |
| Stomach | 0.071 | 0.048 | 0.065 | 0.036 | 0.061 | 0.012 | |
| Urine | 0.686 | 0.656 | 0.469 | 3.167 | 0.604 | 0.118 | |
| Bowl | 0.035 | 0.127 | 0.055 | 0.065 | 0.072 | 0.049 | |
| Muscle | 0.153 | 0.032 | 0.030 | 0.045 | 0.072 | 0.070 | |
| Blood | 0.016 | 0.016 | 0.022 | 0.031 | 0.018 | 0.003 | |
| Heart | 0.126 | 0.013 | 0.062 | 0.097 | 0.067 | 0.057 | |
| Lung | 0.064 | 0.047 | 0.050 | 0.066 | 0.054 | 0.009 | |

TABLE 4
72 h post injection
calculated Total dose = 37043400

| | % of total dose/gm | | | | | | |
|---|---|---|---|---|---|---|---|
| Organ | mouse1 | mouse2 | mouse3 | mouse4 | mean | SD | Mean R/L |
| cord | 0.132 | 0.182 | 0.143 | 0.112 | 0.152 | 0.026 | |
| SN R | 3.109 | 1.020 | 2.493 | 12.680 | 2.207 | 1.074 | 4.600 |
| SN L | 0.374 | 0.044 | 1.021 | 0.649 | 0.480 | 0.497 | |
| Leg R | 7.265 | 5.602 | 6.120 | 5.916 | 6.329 | 0.851 | 7.081 |
| Leg L | 0.799 | 0.972 | 0.910 | 0.960 | 0.894 | 0.088 | |
| liver | 4.035 | 2.072 | 3.094 | 3.753 | 3.067 | 0.982 | |
| Kidney | 8.120 | 9.520 | 2.590 | 18.939 | 6.743 | 3.664 | |
| Spleen | 2.476 | 2.935 | 7.959 | 2.525 | 4.457 | 3.042 | |
| Thyroid | 1.877 | 1.716 | 0.960 | 0.820 | 1.518 | 0.490 | |
| Stomach | 1.194 | 1.395 | 0.000 | 0.876 | 0.863 | 0.754 | |
| Urine | 0.496 | 1.101 | 0.000 | 0.313 | 0.532 | 0.551 | |
| Bowl | 0.614 | 1.466 | 0.783 | 1.254 | 0.954 | 0.451 | |
| Muscle | 0.294 | 0.680 | 0.818 | 0.993 | 0.597 | 0.271 | |
| Blood | 0.864 | 0.300 | 0.181 | 0.210 | 0.448 | 0.365 | |
| Heart | 0.823 | 0.706 | 0.070 | 0.881 | 0.533 | 0.405 | |
| Lung | 1.543 | 1.517 | 1.291 | 1.419 | 1.451 | 0.139 | |

TABLE 5
Summary

| | Time Point | | | | | |
|---|---|---|---|---|---|---|
| | 4 h | | 24 h | | 72 h | |
| Organ | Mean | SD | Mean | SD | Mean | SD |
| cord | 0.017 | 0.014 | 0.014 | 0.009 | 0.152 | 0.026 |
| SN R | 14.223 | 13.849 | 6.972 | 4.603 | 2.207 | 1.074 |
| SN L | 0.088 | 0.104 | 0.019 | 0.018 | 0.480 | 0.497 |
| Leg R | 4.135 | 1.869 | 2.421 | 0.896 | 6.329 | 0.851 |
| Leg L | 0.036 | 0.020 | 0.229 | 0.311 | 0.894 | 0.088 |
| liver | 0.252 | 0.100 | 0.681 | 0.373 | 3.067 | 0.982 |
| Kidney | 1.552 | 0.437 | 1.509 | 1.303 | 6.743 | 3.664 |
| Spleen | 0.133 | 0.028 | 0.246 | 0.087 | 4.457 | 3.042 |
| Thyroid | 0.017 | 0.000 | 0.014 | 0.000 | 1.518 | 0.490 |
| Stomach | 0.784 | 1.296 | 0.061 | 0.012 | 0.863 | 0.754 |
| Urine | 3.723 | 5.441 | 0.604 | 0.118 | 0.532 | 0.551 |
| Bowl | 0.022 | 0.012 | 0.072 | 0.049 | 0.954 | 0.451 |
| Muscle | 0.165 | 0.218 | 0.072 | 0.070 | 0.597 | 0.271 |
| Blood | 0.174 | 0.100 | 0.018 | 0.003 | 0.448 | 0.365 |
| Heart | 0.066 | 0.012 | 0.067 | 0.057 | 0.533 | 0.405 |
| Lung | 0.082 | 0.025 | 0.054 | 0.009 | 1.451 | 0.139 |

TABLE 6
Right/Left Ratios

| Nerves | Mouse1 | Mouse2 | Mouse3 | Mouse4 | Mean | SD |
|---|---|---|---|---|---|---|
| 4 h | 659 | 20 | 136 | 50 | 216 | 299 |
| 24 h | 1180 | 404 | 151 | 845 | 645 | 458 |
| 72 h | 8 | 23 | 2 | 20 | 13 | 10 |

| Legs | Mouse1 | Mouse2 | Mouse3 | Mouse4 | Mean | SD |
|---|---|---|---|---|---|---|
| 4 h | 499 | 59 | 72 | 92 | 180 | 213 |
| 24 h | 31 | 3 | 88 | 79 | 51 | 40 |
| 72 h | 9 | 6 | 7 | 6 | 7 | 1 |

Development of a Nerve Tracking Compound (NTC) and Nuclear and Optical Imaging Study

The base protein (TTC) was purified, and labeled with NHS-DOTA-¹¹¹Indium for nuclear imaging studies and with NHS-Alexa488 or NHS-Alexa688 for optical imaging studies. NTC was injected into the soleus muscle of C57bl mice, and nuclear SPECT-CT imaging performed with the GammaMedica Xspect device, optical in vivo imaging was performed with the Mauna Kea Cell-Vizio LSU-488 system using a S-300-5.0 Proflex fiberoptic probe and the Xenogen IVIS 200 Fluorescent imager, while ex vivo microscopy was performed with the Olympus laser scanning confocal microscope and with an epifluorescence microscope. Bio-distribution studies and histological studies were undertaken. The studies indicated that NTC was taken up in the sciatic nerve after intramuscular injection into the soleus muscle. SPECT-CT images showed distribution along the nerve, confirmed by bio-distribution studies, which demonstrated 6.97±4.6% ID/g (mean±SD) in the ipsilateral sciatic, which was 363 fold higher than the contralateral non-injected side at 24 hours after injection. In vivo optical imaging demonstrated uptake in the sciatic nerve, while histological studies of excised nerve segments confirmed uptake in nerve fassicles within the sciatic nerve. Pharmacokinetic 2-compartment modeling yielded t1/2alpha=1.1 h and t1/2 beta=95.7 h (75.3% and 24.7% contribution respectively). Therefore, labeled NTC is taken up into motor nerve endings after intramuscular injection, and is retrogradely transported in axons. This process is traceable using multiple imaging technologies, and may be useful in the evaluation and treatment of nerve diseases.

Real Time Examination of Alexa488-TTC Sciatic Nerve Distribution

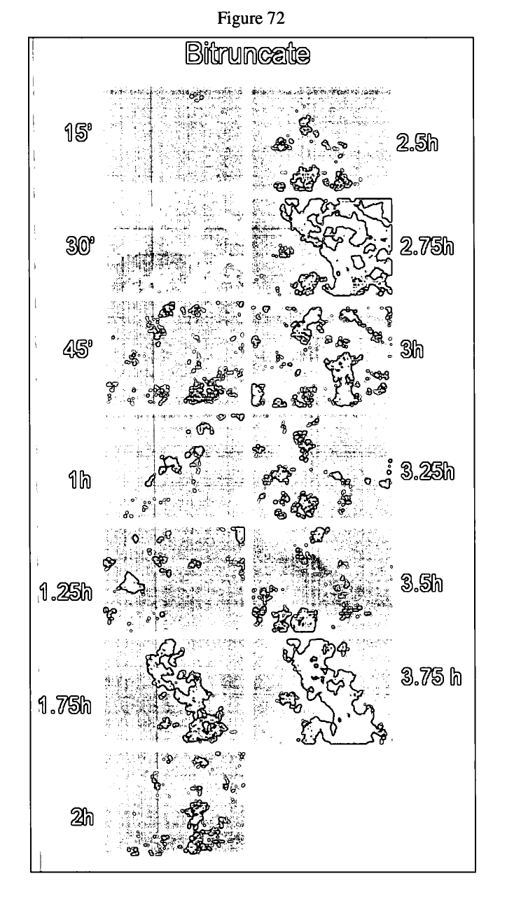

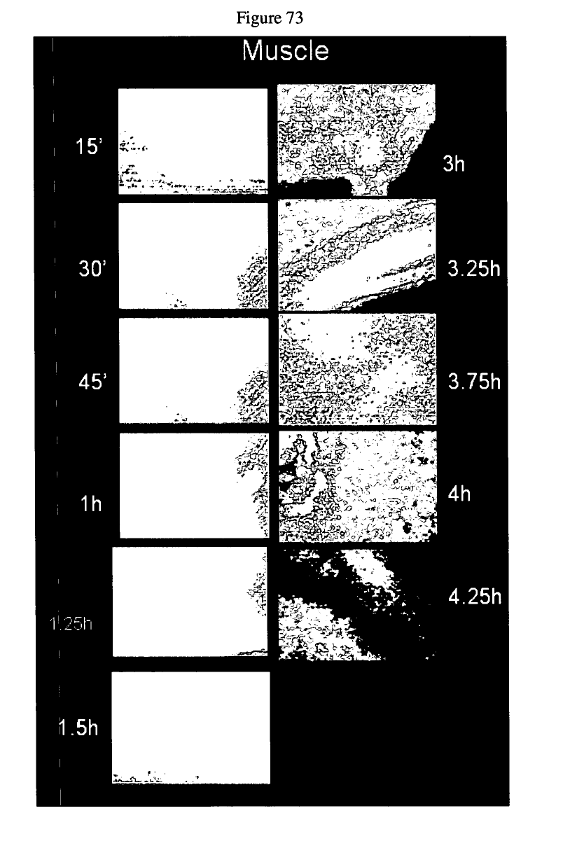

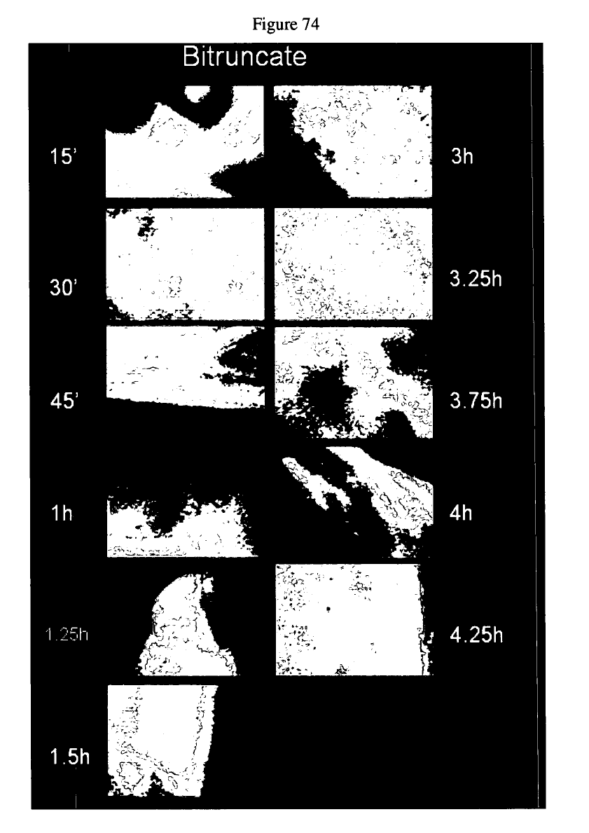

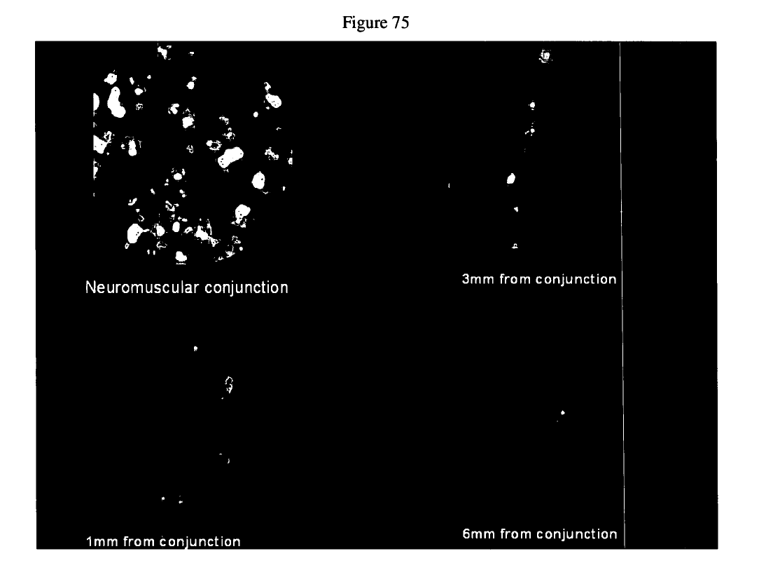

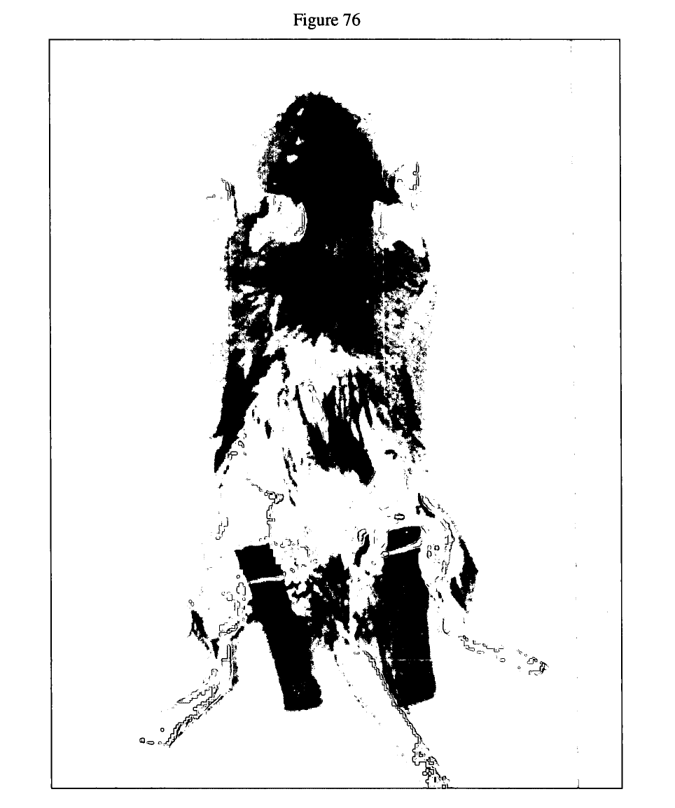

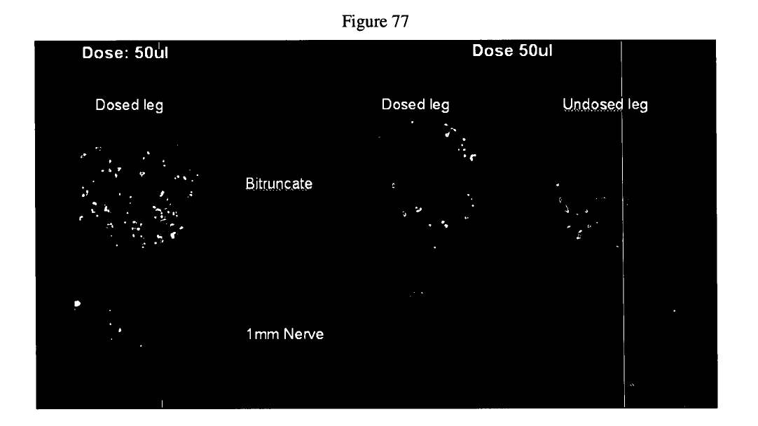

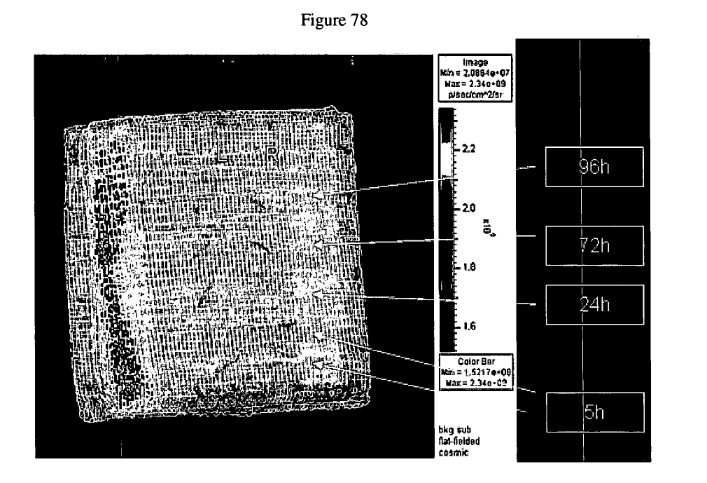

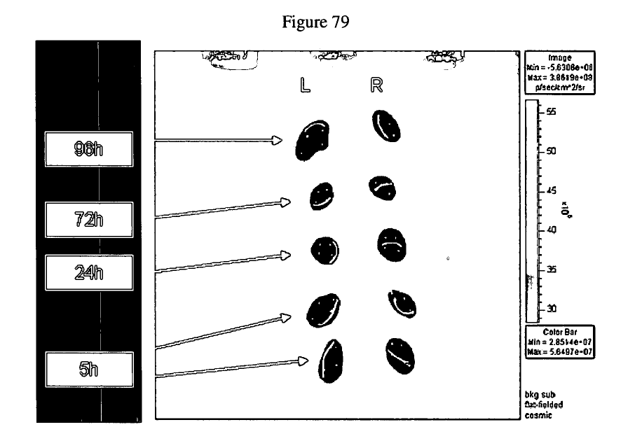

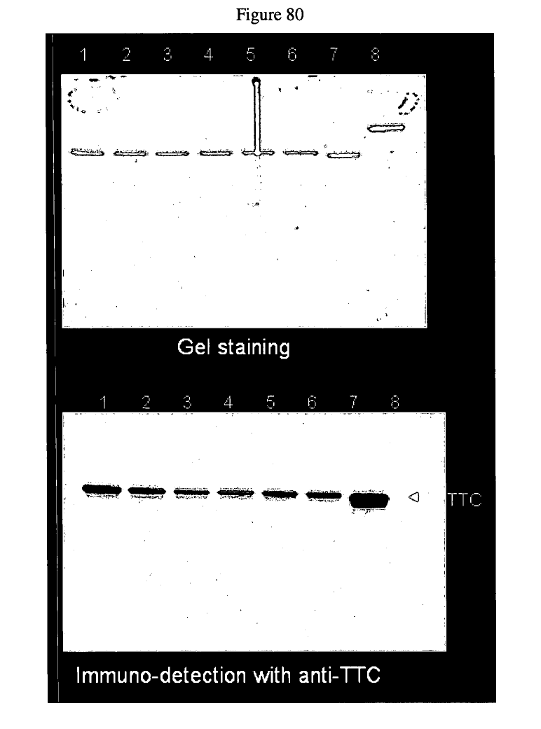

C75BL6 mice were injected with 15 uL or 50 uL of 1.5 mg/ml Alexa488-TTC in the gastrocnemius. The mice were anesthetized with isofluorane at various time points, ranging from 15 minutes to 4.25 hours, and the sciatic nerves were opened for imaging, as shown in FIG. 72-FIG. 73 (15 uL dose) and FIG. 74-FIG. 75 (50 uL dose). Further imaging was conducted with an imaging probe (FIG. 76) at and near the neuromuscular conjunction, as well as CellVizio imaging of the whole mouse receiving the 50 uL injection (FIG. 77) 24 hours after the injection. Similar probe and CellVizio imaging was conducted at 72 and 96 hours after injection (FIG. 78-FIG. 80).

Molecular Imaging of Tumor Spheroids for Screening of Novel Inhibitors of HIF1alpha Signaling.

Hypoxia plays a major role in tumor progression, tumor angiogenesis, and resistance to chemo- and radiotherapy. Hypoxia inducible factor-1α (HIF-1α) is an important regulator of the molecular signaling mechanisms involved in the response to hypoxia. Drugs capable of blocking HIF-1α may be very efficient for anticancer therapy. The goal of this investigation was to assess which of the novel drugs with different mechanisms of action may inhibit or potentiate the inhibition of HIF-1α expression and activity in tumor cell spheroids under hypoxia.

The image analysis software developed in this study would provide 360° average fluorescence intensity profile from the center of spheroid towards the outer edge of the spheroid. This digital tool was used to analyze 3D multi-cellular spheroids of tumor cells bearing HIF-1α-specific dual fluorescence protein reporter system.

The C6#4 reporter cell line constitutively expresses DsRed2/XPRT reporter fusion protein and HIF1α-inducible HSV1tk/GFP fusion reporter protein. Hypoxic core in spheroids of C6#4 cells developed after spheroids grew to more than 350 um in size, as visualized by dynamic quantitative confocal fluorescence microscopy system FV1000 (Olympus) (FIG. 81). A more profound and uniformly distributed hypoxia in these spheroids was achieved by cultivation in medium with 200 μm CoCl2. The level of DsRed2XPRT and HSV1tkGFP expression was determined with a microplate fluorescence spectrometry system (SAFIRE, Tecan). Seventeen drugs with different mechanisms of action were used at different concentrations and in different combinations. Cell viability and proliferation was assessed with WST-1 assay. Individual drugs of combinations that did not decrease cell viability, but decreased HIF1α levels or HIF1α-inducible transcriptional activity were identified. From 17 drugs tested in this investigation, ten suppressed CoCl2-induced HIF1α signaling with different potency, including: PX-478, Arctigenin, LY 294002, Iressa, Tarceva, Orlistat, Edelfosine, Gemzar, Valcade, and Anisomycin. Seven other drugs had no significant effect on HIF1α signaling, including: Indirubin, Deguelin, Gleevec, PD 168393, Erbitux, SB 203580, and Rapamycin. In C6#4 spheroids, PX-478 inhibited the level of HIF1α expression and activity, HIF1α signaling was also down-regulated by inhibitors of EGFR kinase and P13K, but not by putative inhibitors of Akt and mTOR signaling.

Spheroids grow larger over time; their centers gradually become hypoxic, as indicated by the induction of the HIF1-alpha pathway visualized by the expression of GFP. Subjecting spheroids to hypoxic experimental conditions (Cobalt chloride) rapidly induces hypoxia in the entire spheroid within 6-8 hours, while untreated spheroids developed hypoxic cores after about 3 days in culture. This hypoxic response is inhibited by a Hif 1-alpha inhibitor, PX 478. Cellular motility is affected by hypoxia, and is currently under study. Prior to the methods of the present disclosure, the analysis of the spheroids were being done based on the overall intensity values and manually extracting radial profiles. In practice, this is prohibitively expensive of labor and not feasible to complete for a large numbers of spheroids.

Quantitation of Spatial and Temporal Dynamics of Expression of Fluorescent Reporter Proteins in Multi-Cellular Tumor Spheroids

Custom software was written to perform average radial profile image analysis on the user specified central image slice through the spheroid. The RFP channel was used to threshold the data and to determine the center of the spheroid. Using this computer determined center as a fulcrum, a radial arc was swept through user specified 360 degrees, while plotting a GFP and RFP expression plot profile along each radius (plot line thickness=1 pixel). Microscopy imaging datasets (Olympus FV-1000) included constitutively expressed RFP and HIF-1α-inducible GFP channels acquired at 20 μm intervals using a 800×800 imaging matrix/image for a typical imaging stack of 12 images/spheroid over 5-7 days. Image datasets were analyzed with the new software and displayed as GFP/RFP intensity ratio as a function over a distance along the maximum radius. Spheroids of 710±20 um in diameter developed within 3 days a “ring-shaped” hypoxic area with a peak of HIF-1α-induced GFP fluorescence at 120±30 um from the spheroid center. Over the following 3 days, this hypoxic ring gradually extended towards spheroid periphery, with stellar-like extensions towards spheroid periphery and increased fluorescence intensity, reflecting pathways of hypoxic cell migration. Spheroid border was populated with several layers of highly GFP-positive cells with persistent HIF-1α signaling activity. The newly developed software tool for measurement of average radial fluorescence intensity profiles in confocal fluorescence microscopy images of 3D spheroids and allows for quantitative characterization of spatial heterogeneity and temporal dynamics of fluorescence distribution within multi-cellular 3D spheroids (FIG. 89).

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for purifying Tetanus Toxin Fragment C comprising obtaining a supernatant comprising soluble Tetanus Toxin Fragment C and purifying Tetanus Toxin Fragment C under native conditions to obtain a substantially purified Tetanus Toxin Fragment C.

2. The method of claim 1, wherein the substantially purified Tetanus Toxin Fragment C is biologically active.

3. An imaging agent comprising a Tetanus Toxin Fragment C and a reporter.

4. The imaging agent of claim 3, wherein the Tetanus Toxin Fragment C is a substantially purified Tetanus Toxin Fragment C.

5. The imaging agent of claim 3, wherein the reporter is a fluorescent label or a radio label.

6. The imaging agent of claim 3 further comprising a therapeutic moiety selected from the group consisting of a drug, a growth factor, a radiation emitting compound, and any combination thereof.

7. A method comprising introducing an imaging agent comprising a Tetanus Toxin Fragment C and a reporter into a mammal, and detecting a signal in the mammal from the imaging agent.

8. The method of claim 7, wherein the signal is detected using one or more of magnetic resonance imaging, positron emission tomography, and computed tomography imaging.

9. The method of claim 7, wherein the imaging agent further comprises a therapeutic moiety selected from the group consisting of a drug, a growth factor, a radiation emitting compound, and any combination thereof.

10. A method comprising processing confocal microscopy datasets to provide a 360 degree average fluorescence intensity profile from the center of a spheroid towards the outer edge of the spheroid.

11. The method of claim 10, wherein the spheroid is a tumor spheroid, or portion thereof.

12. The method of claim 10, further comprising inputing data that represents a confocal microscopy dataset, reading in an image, converting the image to grey-scale, determining a binary threshold, filing holes in the image, selecting the image over background, determining center of the image, selecting an outline of the image, determining an average radius of the image, calculating a radial intensity profile of the image, saving the profile data, and importing the profile data into a graphing program.

13. A system comprising: a first storage medium including data that represent a confocal microscopy dataset; a program capable of processing confocal microscopy datasets to provide a 360 degree average fluorescence intensity profile; and a processor capable of executing the program.
